(19) United States
(12) Patent Application Publication
(10) Pub. No.: US 2025/0182638 A1
(43) Pub. Date: Jun. 5, 2025

(54) METHODS AND APPARATUS FOR ENHANCING HUMAN COGNITION

(71) Applicant: Vimbal Enterprises PTY LTD, Adelaide (AU)

(72) Inventor: Joshua Peter Arnall, Yarram, Victoria (AU)

(21) Appl. No.: 18/877,926

(22) PCT Filed: Jun. 22, 2023
(86) PCT No.: PCT/AU2023/050573

§ 371 Date: Dec. 20, 2024

(30) Foreign Application Priority Data
Jun. 22, 2022 (AU) 2022901721

(51) Int. Cl. G09B 5/06; G06F 40/205; G06T 11/00; G16H 40/67

(52) U.S. Cl. CPC G09B 5/06; G06F 40/205; G06T 11/001; G16H 40/67

(57) ABSTRACT

This disclosure is of a human sensory information presentation arrangement for presenting sensory information to a human having a controller programmed to make available human visual and auditory presentation information, including at least visual information and non-verbal auditory signals; at least one human visual presentation device for receiving visual information from the controller, wherein the visual information comprises at least one or more human-readable characters or structured set of characters from a predetermined alphabet and predetermined number set to form a human-readable word or a number, presented one character or one structured set of characters at a time; and at least one human auditory presentation device for receiving a non-verbal auditory signal from the controller, and wherein the controller presents each human-readable character or structured set of characters while simultaneously presenting at least one of a predetermined non-verbal auditory signal. The arrangement can be used by various users with different mental acuity and cognitive capabilities, and certain combinations of elements and methods of use will be more effective than others in training, reading tasks, and assisting those with various human cognitive disorders and conditions.

### FIELD OF THE DISCLOSURE

The field of this disclosure is methods, apparatus and systems for enhancing human cognition, the mental action or process of acquiring knowledge and understanding through thought, experience and the senses.

### BACKGROUND

Humans learn to survive and, in most cases, thrive in their environment. The act of learning is multi-faceted, and the field of cognitive neuropsychology has evolved from early recognition by humans that humans do things in and react to their environment in primarily predictable ways. However, learning is not a process that is the same for all humans, even if, statistically, there is a normal distribution of how humans can receive and process information and then create relationships between perceived events and knowledge about that event. Even remembering that event, the ability to recall that event and to understand what that event means in the context of the event at the time or in the future are very human traits.

Perception is at the core of learning, and most humans have five senses (taste, smell, sight, touch, and hearing), which facilitate observation and awareness of the human environment. However, not all humans have the same abilities to use each sense.

Many studies indicate that some humans are visual learners. First, they see, and then they can understand and do. Others need to be told, and others need both, being told and shown what to do. When touch is part of the event, some use touch better than others, and when the event includes taste and smell, some humans will excel at one or both for receiving those sensory inputs and associating them with concepts and facts.

Some humans consciously or unconsciously inhibit or attenuate their understanding of their perceptions. Consequently, their recall of sensorial experiences is affected due to psychological issues common to humans and some issues that are unique to the individual.

Learning is one of the multiple outcomes of receiving sensory input into the mind of a human, and those inputs can fill every waking moment and even be perceived while sleeping. First, the human sensory input mechanisms transform the sensory inputs into perceived information, and after some processing, all or some of that information is stored in memory locations. After that, the brain exercises its ability to reason using the available information and sometimes still incoming perceived sensory inputs. Reasoning allows the vast array of memorised information to be used, and logical treatment of that information is one example of the human brain's thinking and cognition capabilities that contribute to learning.

The study of how humans learn is a field of great interest. It is fundamental to the human experience, but more so when humans need to learn to pass a test or be deemed fit to practice a skill or to work within an environment safely and with responsibility. There are so many more areas of human endeavour, and for humans to accept and learn new information during their work and private life means that improvements in how humans learn will benefit them and others. How humans perceive information is vital to the effectiveness of learning from that information.

However, it is typical for the learning process to involve only one of the human senses at a time. This restricts the learning capability of most humans. When more than one sense is involved, there are many examples of one sensory input being compromised by the simultaneous reception of another sensory input. There is scope for improvements in presenting visual and auditory sensory input to a human to achieve more effective retention and cognition of information than prior approaches.

### Aspects of the Disclosure

In an aspect a human sensory information presentation arrangement for presenting sensory information to a human, the arrangement comprises: a controller comprising a memory and a central processing unit programmed to make available human visual and auditory presentation information, including at least visual information and non-verbal auditory signals; at least one human visual presentation device for receiving visual information from the controller, the visual presentation device having a configuration that exclusively displays the received visual presentation information to the one human within a predetermined field of view, wherein the visual information comprises at least one or more human-readable characters or structured set of characters from a predetermined alphabet and predetermined number set to form a human-readable word or a number, presented one character or one structured set of characters at a time; and at least one human auditory presentation device for receiving a non-verbal auditory signal from the controller, the human auditory presentation device having a configuration that exclusively directs the non-verbal auditory signal to the one human, the non-verbal auditory signal comprising one or more non-verbal auditory signals, wherein the controller presents each human-readable character or structured set of characters while simultaneously presenting at least one of a predetermined non-verbal auditory signal.

In an aspect, the presentation of the human-readable characters or structured set of characters are presented by the visual presentation device to contrast with a predetermined background colour also presented by the visual presentation device.

In an aspect, the visual presentation of a human-readable character or structured set of characters includes one or more of the group: a predetermined colour for one or more of the human-readable characters or the structured set of characters; a grouping of different coloured human-readable characters or structured set of characters; the human-readable character or structured set of characters that are adapted to appear to move in front of the human viewing the presentation.

In an aspect, a predetermined colour is used for a predetermined word and to contrast with a predetermined different colour to that of the predetermined word.

In an aspect, predetermined colours are used when a series of presented words form a human-readable phrase or a human-readable sentence.

In an aspect, the predetermination of colour palettes, words, phrases, sentences or font size involves the use of a library of such elements that can be used to source the required element. So, for example, when a certain colour palette is to be used that suits an autistic human user, that colour palette is predetermined by input provided at the beginning of the user session, made from choices made available which are recorded and used by the server computer or the local processor. Likewise, using predetermined colours for text and background visual information presentation. In another example. When a word is predetermined, the word is sourced from memory associated with the computer server or the local processor. That source is the result of the parsing of the source information that is to be presented during a session, wherein a selection of the sources and, thus, the visual information is to be presented for learning during a session at the beginning of that session for the human user, sa, a safety course recipient, or a human wanting to read a particular book, etc. Likewise, with the use of predetermined phrases, sentences or font size. The later-mentioned font size can be predetermined to suit a particular human with visual acuity issues.

In an aspect, the visual presentation comprises the human-readable character or structured set of characters that appear to move close to the human and then further from the human within their field of view, wherein the movement coincides with the appearance of each successive human-readable character or structured set of characters or during the appearance of a successive human-readable character or structured set of characters.

In an aspect, the rate of the successive presentation of a human-readable character or structured set of characters presented to the human is changeable from an initial predetermined rate.

In an aspect, the font size of the successive presentation of a human-readable character or structured set of characters presented to the human is changeable from an initial predetermined font size.

In an aspect, the successive presentation of a human-readable character or structured set of characters presented to the human with an image of a trap door and the prior human-readable character or structured set of characters presented to the human appears to enter and disappear into the trap door.

In an aspect, the successive presentation of a human-readable character or structured set of characters presented to the human with an image of a moving corridor and the prior human-readable character or structured set of characters presented to the human appears to enter and disappear into the corridor.

In an aspect, the successive presentation of a human-readable character or structured set of characters is presented to the human with repetition of one or more of the human-readable character or structured set of characters.

In an aspect, each predetermined non-verbal auditory signal is continuous during the presentation of a human-readable character or structured set of characters and between the successive presentation of a human-readable character or structured set of characters.

In an aspect, the predetermined non-verbal auditory signal is binaural and provided to the respective ears of the human.

In an aspect, a predetermined non-verbal auditory signal is a combination of two tones, and monaural tones are provided to both human ears.

In an aspect wherein the predetermined auditory signal is binaural and provided to the respective ears of the human.

In an aspect, a predetermined non-verbal auditory signal is isochronic tones. In an aspect, the isochronic tones have a predetermined pitch and predetermined interval, and in an aspect, at least one human auditory presentation device for receiving a non-verbal auditory signal from the controller further comprises a transducer to convert electrical energy, representative of a non-verbal auditory signal controlled and provided by the controller, into mechanical energy to vibrate the surrounding air, the transducer being located near an ear of the human, the received vibrated air being a representation of the non-verbal auditory signal.

In an aspect, at least one human auditory presentation device further comprises a housing having the transducer located internal to the casing, wherein the housing is adapted to direct sound generated by the non-verbal auditory transducer into the ear of the human.

In an aspect, the presentation of human-readable characters or structured sets of characters is coordinated with the presentation of a predetermined non-verbal auditory signal by an application programming interface executed by the controller.

In an aspect, the human visual presentation device comprises one video signal presentation screen or two video signal presentation screens that extend at least to the boundary of the field of view of the human.

In an aspect, a computer server having a computer server memory and a central processing unit adapted to make available visual and non-verbal auditory presentation information from the computer server memory; a computer device having a digital signal receiving arrangement, a computer device memory and a central processing unit adapted to store an application programming interface in the computer device memory and execute the application programming interface, which is adapted to receive and process visual and non-verbal auditory presentation information made available by the computer server; and at least one human visual presentation device for receiving visual information from the computer device, the visual presentation device having a configuration that exclusively displays the received visual presentation information to the one human user within a predetermined field of view, wherein the visual information comprises at least one or more human-readable characters or structured set of characters from a predetermined alphabet and predetermined number set to form a human-readable word or a number, presented one character or one structured set of characters at a time; and at least one human auditory presentation device for receiving a non-verbal auditory signal from the computer device, the human auditory presentation device having a configuration that exclusively directs the non-verbal auditory signal to the one human user, the non-verbal auditory signal comprising one or more non-verbal auditory signals, wherein the computer device presents each human-readable character or structured set of characters while simultaneously presenting at least one of a predetermined non-verbal auditory signal.

In an aspect, the visual presentation information is made available to the computer server and stored in the computer server memory.

In an aspect, the central processor parses visual presentation information stored in the computer server memory being partitioned to identify words and sentences or subsets of a complete sentence using one or more text spacing elements or punctuation symbols as the delimiter of the word or sentence or a subset of a complete sentence.

The system, methods and apparatus disclosed in this specification are intended to provide at least an alternative to any systems currently available and alleviate or minimise their problems and shortcomings.

The reference to any prior art in this specification is not and is not to be taken as an acknowledgement of any form of suggestion that such prior art forms part of the common general knowledge.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers. The term “a” or “an” means “one or more” unless the context indicates otherwise.

It will be appreciated by those skilled in the art that the disclosure described herein is not restricted in its use to the particular application/s described. Neither is the disclosure restricted in any preferred embodiment concerning the elements and features described or depicted herein. It will be appreciated that the scope of the disclosure is not limited to the embodiment or embodiments disclosed but is capable of numerous rearrangements, modifications, and substitutions without departing from the scope set forth and defined by the claims.

Some embodiments described herein may be implemented using programmatic elements, often called modules or components, although other names may be used. Such programmatic elements may include a program, a subroutine, a portion of a program, a software component, or a hardware component capable of performing one or more stated tasks or functions. As used herein, a module or component can exist on a hardware component independently of other modules/components, or a module/component can be a shared element or process of other modules/components, programs or machines. A module or component may reside on one machine, such as a client or a computer server. A module/component may be distributed amongst multiple machines, such as on multiple clients or computer server machines. Any system described may be implemented in whole or in part on a computer server or as part of a network service. Alternatively, a system described herein may be implemented on a local computer, terminal, or server in whole or in part. In either case, implementation of the system provided for in this application may require using memory, processors and network resources (including data ports and signal lines (optical, electrical and other communication modalities), unless stated otherwise.

Some embodiments described herein may generally require computers, including processing and memory resources. For example, systems described herein may be implemented on a server or network. Such computer servers may connect and be used by users over networks such as the Internet or by a combination of networks, such as cellular networks and the Internet. Alternatively, one or more embodiments described herein may be implemented locally, in whole or in part, on computing machines such as desktops, cellular phones, personal digital assistances or laptop computers. Thus, memory, processing and network resources may be used in connection with the establishment, use or performance of any embodiment described herein (including the performance of any method or the implementation of any system).

Furthermore, some embodiments described herein may be implemented using instructions that are executable by one or more processors. These instructions may be carried on a computer-readable medium. Machines that may be shown in the figures provide examples of processing resources and computer-readable mediums on which instructions for implementing embodiments can be carried out and executed. The numerous machines associated with one or more embodiments include a processor(s) and various forms of memory for holding data and instructions. Examples of computer-readable mediums include permanent memory storage devices, such as hard drives on personal computers or computer servers. Other examples of computer storage mediums include portable storage units, such as CD or DVD units, flash memory (carried on many cell phones and personal digital assistants (PDAs), and magnetic memory. Computers, terminals, and network-enabled devices (e.g. mobile devices such as cell phones) are all examples of processors and devices. Instructions are usually stored on transitory and non-transitory computer-readable mediums, including RAM, ROM and EPROM devices.

The present disclosure can be implemented in numerous ways, including as a process, an apparatus, a system, or a computer-readable medium such as a computer-readable storage medium or a computer network wherein program instructions are sent over wireless, optical, or electronic communication links. It should be noted that the order of the steps of disclosed processes may be altered within the scope of the disclosure.

Details concerning computers, computer networking, software programming, telecommunications, and the like may, at times, are not illustrated explicitly as such was not considered necessary to obtain a complete understanding nor to limit a person skilled in the art of performing the embodiments, are considered present nevertheless as such are considered to be within the skills of persons of ordinary skill in the art.

The prior summary introduces a simplified selection of concepts, further described below in the Detailed Description of Embodiments. The summary does not intend to identify any or all of the claimed subject matter's key or essential features.

It will be appreciated by those skilled in the art that this disclosure is not restricted in its use to the particular application or applications described. Neither is the present disclosure restricted in its preferred embodiment concerning the particular elements and features described or depicted herein. It will be appreciated that the disclosure is not limited to the embodiment or embodiments disclosed but is capable of numerous rearrangements, modifications and substitutions without departing from the scope set forth.

### BRIEF DESCRIPTION OF THE FIGURES

FIG. 1 depicts an application configuration flow chart dealing with text and colour;

FIG. 2 depicts an application configuration flow chart dealing with text-to-speech;

FIG. 3 depicts a visual features process flow chart dealing with selecting and processing user files;

FIG. 4 depicts a word parsing process flow chart dealing with processing and rendering a word;

FIG. 5 depicts a word-per-minute process flow chart dealing with word incrementing processes;

FIG. 6 depicts a word positioning process flow chart dealing with word location within a field of view;

FIG. 7 depicts a selected word and selected colour rendering process;

FIG. 8 depicts a selected word and selected colour of FIG. 7 display process;

FIG. 9 depicts a selected word and selected colour display process of FIG. 8 and applying automatic speed ramping;

FIG. 10 depicts a selected word and selected colour display process, applying an automatic speed ramping process of FIG. 9 and font size adjustment;

FIG. 11 depicts a selected word and selected colour display process, applying automatic speed ramping; font-size adjustment process of FIG. 10 and location adjustment within the field of view;

FIG. 12 depicts a selected word and selected colour display process, applying automatic speed ramping, font size adjustment, location adjustment within the field of view process of FIG. 11 and font adjustment to accommodate a dyslexic user,

FIG. 13 depicts a word parsing process flow chart dealing with processing and rendering a word in colour;

FIG. 14 depicts a word parsing process flow chart dealing with processing and rendering a word in the colour process of FIG. 13 and displays one word at a time;

FIG. 15 depicts a word parsing process flow chart dealing with processing and rendering a word in colour and displaying one word at a time process of FIG. 14 and applying automatic speed ramping;

FIG. 16 depicts a word parsing process flow chart dealing with processing and rendering a word in colour, displaying one word at a time, and applying the automatic speed ramping process of FIG. 15 and font adjustment;

FIG. 17 depicts a word parsing process flow chart dealing with processing and rendering a word in colour, displaying one word at a time, and applying automatic speed ramping and font adjustment process of FIG. 16 and location adjustment within the field of view;

FIG. 18 depicts a word parsing process flow chart dealing with processing and rendering a word in colour, displaying one word at a time, and applying automatic speed ramping, font adjustment and location adjustment within the field of view process of FIG. 17 and font adjustment to accommodate a dyslexic user;

FIG. 19 depicts a word parsing process flow chart dealing with processing and rendering a word in colour to accommodate a dyslexic user;

FIG. 20 depicts a word parsing process flow chart dealing with processing and rendering a word in colour to accommodate a dyslexic user process of FIG. 19 and display one word at a time;

FIG. 21 depicts a word parsing process flow chart dealing with processing, rendering a word in colour to accommodate a dyslexic user and displaying one word at a time process of FIG. 20 and applying automatic speed ramping;

FIG. 22 depicts a word parsing process flow chart dealing with processing, rendering a word in colour to accommodate a dyslexic user, displaying one word at a time and applying the automatic speed ramping process of FIG. 21 and font adjustment;

FIG. 23 depicts a word parsing process flow chart dealing with processing, rendering a word in colour to accommodate a dyslexic user, displaying one word at a time, applying automatic speed ramping and font adjustment process of FIG. 22 and location adjustment within the field of view;

FIG. 24 depicts a word parsing process flow chart dealing with processing, rendering a word in colour to accommodate a dyslexic user, displaying one word at a time, applying automatic speed ramping, font adjustment and location adjustment within the field of view process of FIG. 23 and font adjustment to accommodate a dyslexic user;

FIG. 25 depicts an environment creation apparatus using a dark display background for a single word;

FIG. 26 depicts an environment creation apparatus using a dark display background for a single word, as in FIG. 25 using a corridor display background;

FIG. 27 depicts an environment creation apparatus using a dark display background for a single word using a corridor display background of FIG. 26 and location adjustment within the field of view;

FIG. 28 depicts an environment creation apparatus using a dark display background for a single word using a corridor display background, and location adjustment within the field of view of FIG. 27 and using a raised line of sight within the field of view;

FIG. 29 depicts an environment creation apparatus using a dark display background for a single word using a corridor display background, location adjustment within the field of view and using a raised line of sight within the field of view of FIG. 28 and an adjustment of the word representation to facilitate disassociated learning including a display of the word from side-on concerning the user's field of view;

FIG. 30 depicts an environment creation apparatus using a dark display background for a single word using a corridor display background, location adjustment within the field of view, using a raised line of sight within the field of view and an adjustment of the word to facilitate disassociated learning of FIG. 29 and an adjustment of the word representation to facilitate a trap door visualisation;

FIG. 31 depicts an environment creation apparatus using a dark display background for a single word using a corridor display background, location adjustment within the field of view, using a raised line of sight within the field of view, an adjustment of the word to facilitate disassociated learning and an adjustment of the word representation to facilitate a trap door visualisation of FIG. 30 with the user experience being stopped at a random interval;

FIG. 32 depicts a selected word and selected colour rendering process advantageous for a neurodiverse user;

FIG. 33 depicts a selected word and selected colour rendering process advantageous for a neurodiverse user process of FIG. 32 and using a corridor display background;

FIG. 34 depicts a selected word, selected colour rendering process advantageous for a neurodiverse user and using a corridor display background process of FIG. 33 and location adjustment within the field of view of the user;

FIG. 35 depicts a selected word, selected colour rendering process advantageous for a neurodiverse user, using a corridor display background and location adjustment within the field of view process of FIG. 34 and using a raised line of sight within the field of view;

FIG. 36 depicts a selected word, selected colour rendering process advantageous for a neurodiverse user, using a corridor display background, location adjustment within the field of view and using a raised line of sight within the field of view process of FIG. 35 and an adjustment of the word representation to facilitate disassociated learning including a display of the word from side-on concerning the user's field of view;

FIG. 37 depicts a selected word, a selected colour rendering process advantageous for a neurodiverse user, using a corridor display background, location adjustment within the field of view and using a raised line of sight within the field of view and an adjustment of the word representation to facilitate the disassociated learning process of FIG. 36 and an adjustment of the word representation to facilitate a trap door visualisation;

FIG. 38 depicts a selected word, a selected colour rendering process advantageous for a neurodiverse user, using a corridor display background, location adjustment within the field of view and using a raised line of sight within the field of view, an adjustment of the word representation to facilitate disassociated learning and an adjustment of the word representation to facilitate a trap door visualisation process of FIG. 37 and with the user experience being stopped at a random interval;

FIG. 39 depicts a representation of a moving corridor to convey to the user the impression that the user is moving forwards;

FIG. 40 depicts a representation of a moving corridor process of FIG. 39 with location adjustment of the word within the field of view;

FIG. 41 depicts a representation of a moving corridor with location adjustment of the word within the field of view process of FIG. 40 and the use of binaural beats provided into an environment creation apparatus;

FIG. 42 depicts a representation of a moving corridor with location adjustment of the word within the field of view process of FIG. 40 and the use of binaural beats provided into an environment creation apparatus and a prompt to the user to widen their eyes;

FIG. 43 depicts a representation of a moving corridor with location adjustment of the word within the field of view process of FIG. 40, the use of binaural beats provided into an environment creation apparatus, a prompt to the user to widen their eyes and using a raised line of sight within the field of view of the user;

FIG. 44 depicts a representation of a moving corridor with location adjustment of the word within the field of view process of FIG. 40, the use of binaural beats provided into an environment creation apparatus, a prompt to the user to widen their eyes and using a raised line of sight within the field of view of the user with repetition of prior displayed words or sets of words;

FIG. 45 depicts a representation of a moving corridor with location adjustment of the word within the field of view process of FIG. 40, the use of binaural beats provided into an environment creation apparatus, a prompt to the user to widen their eyes and using a raised line of sight within the field of view of the user with repetition of prior displayed words or sets of words and an adjustment of the word representation to facilitate disassociated learning including a display of the word from side-on concerning the user's field of view;

FIG. 46 depicts a representation of a moving corridor with location adjustment of the word within the field of view process of FIG. 40, the use of binaural beats provided into an environment creation apparatus, a prompt to the user to widen their eyes and using a raised line of sight within the field of view of the user with repetition of prior displayed words or sets of words, an adjustment of the word representation to facilitate disassociated learning including a display of the word from side-on concerning the user's field of view and an adjustment of the word representation to facilitate a trap door visualisation;

FIG. 47 depicts a representation of a moving corridor with location adjustment of the word within the field of view process of FIG. 40, the use of binaural beats provided into an environment creation apparatus, a prompt to the user to widen their eyes and using a raised line of sight within the field of view of the user with repetition of prior displayed words or sets of words, an adjustment of the word representation to facilitate disassociated learning including a display of the word from side-on concerning the user's field of view, an adjustment of the word representation to facilitate a trap door visualisation and the user experience being stopped at a random interval;

FIG. 48 depicts a human non-verbal auditory presentation device having a configuration that exclusively directs non-verbal auditory presentation information in the form of a non-verbal auditory signal to the ears of one human using binaural beats having predetermined frequencies and a combination/summing arrangement to add one of a predetermined audio recording;

FIG. 49 depicts a human non-verbal auditory presentation device having a configuration that exclusively directs non-verbal auditory presentation information in the form of a non-verbal auditory signal to the ears of one human using binaural beats having predetermined frequencies and a text-to-speech arrangement to add one of a predetermined audio recording or machine created verbalisation of a predetermined source of text;

FIG. 50 depicts a human non-verbal auditory presentation device having a configuration that exclusively directs non-verbal auditory presentation information in the form of a non-verbal auditory signal to the ears of one human using binaural beats having predetermined frequencies and a combination/summing arrangement to add one of a predetermined audio signal along with a text to speech arrangement to add one of a predetermined audio recording or machine created verbalisation of a predetermined source of text;

FIG. 51 depicts sub-learning sessions where a learning session is broken down into multiple randomly spaced sessions with breaks in between;

FIG. 52 depicts the elements of FIG. 51 plus an added animation to illustrate the trapped door on head imagery;

FIG. 53 depicts the elements of FIGS. 51 and 52 plus an added Eye Movement Desensitization and Reprocessing simulation;

FIG. 54 depicts a calibration process for a head-worn Virtual Reality device and a set-up process depicting the adjustments that may be required if a user has a visual blind spot;

FIG. 55 depicts an embodiment of the elements of a head-worn Virtual Reality device;

FIG. 56 depicts an embodiment of a document upload arrangement and Client Server cloud architecture, and

FIG. 57 depicts sound processing techniques to make the sound seem to have a source remote from the user, as illustrated in front, above and behind, at any one time.

### DETAILED DESCRIPTION OF EMBODIMENTS

The method, apparatus, and system disclosed comprise a visual and non-verbal auditory arrangement for use (by example only) in training personnel working in hazardous environments. In addition, although aspects of the proposed system, either singly or in combination, may be for use in the treatment and management of Post Traumatic Stress Disorder (PTSD), anxiety, Attention-Deficit/Hyperactivity Disorder (ADHA), autism, Dyslexia and other neurological ailments, and also for use in accelerating learnings by students and professionals of knowledge that is typically acquired by reading. Other possible application fields include gaming, retail, sports and any learning or training environment.

The arrangement of elements and characteristics include visuals, including but not limited to the use of contrasting colours/selected colour palettes to account for dyslexia and colour blindness, colourisation of human-readable characters or structured set of characters (hereafter referred to as text which may also be referred to as word) and backgrounds, timing and movement (predominantly of text on a visual presentation device), use of eye movement desensitisation and reprocessing, and use of the perceived forward motion of text as it is being viewed. In addition, in an embodiment, there is the adaption of parts of the arrangement to provide an immersive sensorial distraction-free environment for the human or humans using the arrangement. In an embodiment, a structured set of characters can form a word known in a dictionary of words created out of the subject alphabet. Alternatively, a structured set of characters may represent an acronym, slang, or word yet to be in a dictionary. In a scientific or engineering document, the structured set of characters may be a formula or an abbreviation for a known longer form of the abbreviation. A structured set of characters may be numbers or characters from another alphabet or language. Translating or transforming some structured sets of characters may be necessary to be more likely understood by the user.

The arrangement of elements and characteristics also include audible cues such as text to voice, binaural beats embedded in the delivery of a voice verbalising the displayed text, binaural beats in the background and particular use of left ear delivery of 111 Hz sound. In contrast, the right ear delivers beats within selected bands that affect the human in a range of respective states such as relaxation (other suitable singular frequencies can be used), the equivalent state to rapid eye movement, focused attention and high-level engagement. The preceding disclosed audible cues are provided with or without one or more visual cues previously described in a preliminary preparatory phase, intermediate the beginning and end of the process. As well as afterwards, the process allows a person to process the text information they have perceived, but in an embodiment that does not apply to text-to-voice cues.

The reference to the human-readable character or structured set of characters includes but is not limited to characters that are a subset of a corresponding alphabet. A character or a structured set of characters may represent a single character, word or collective character symbol or have a known meaning. Also, a structured set of characters may represent a word whose meaning is known, or the set of characters may collectively represent an image with the meaning being known. Sometimes the preceding or following character/s or structured set of characters will contribute to the meaning of the character or structured set of characters being presented to the human. Visual information is a human-readable character or structured set of characters of one or more alphabets.

The methods, apparatus and system disclosed will create an arrangement that scales from the minimum essential cues to affect a person using the arrangement to a combination of cues that deliver to a selected person or group of a particular type of person, wherein there may be variations depending on the types of user or the type of users within a group.

Generation of and control of the visual and audible cues is under computer control, such as a computer server and associated Application Programming Interface that can be delivered from a cloud-based computing environment and access to users available on a time and specific purpose-based Software as a Service basis. The user may supply a source text library or image library, or there may be preselected text and image libraries that become part of the available service.

Isochronic tones are single tones presented to the listener on and off at regular, evenly-spaced intervals. The interval is typically brief, creating a beat like a rhythmic pulse. Such sounds can be embedded in other sounds, such as music or natural sounds.

A binaural stereo headset has two channels and two speakers. With a stereo headset, control is always asymmetrical, i.e. two different signals output on the two loudspeakers but both at the same volume.

A binaural splitting headset has two completely separate channels. In addition, each speaker has a separate channel so that the signals can be output independently, meaning each speaker can have a different volume. It is also possible for the two signals to be provided in only one speaker or both speakers, which can be used for those users that prefer or need one ear use.

In an aspect of the arrangement, visual and non-verbal auditory presentation information is provided with an immersive sensory distraction-free environment for the human or multiple humans. This aspect intends to remove as much external stimulus that is about the human as possible to provide an immersive environment. A Virtual Reality (VR) headset is one embodiment of such an arrangement wherein the VR device has a structure that isolates the human from external visual and audible input and permits only predetermined visual and predetermined non-verbal auditory presentation information using a dedicated visual presentation device. The visual presentation device, by way of example, comprises one video signal presentation screen or two video signal presentation screens (one for each eye of the human or a screen viewed by both eyes of the human user). One video signal presentation device comprises a screen or two visual information presentation screens located within the headset and exclusively displays the received visual presentation information to the human wearer of the headset. The screen or screens are sized to provide viewing by the human within the boundary of the field of view of the human wearing the headset. The headset is configured to exclude the human from viewing anything but the video signal presentation device or devices. The resolution, frames per second, luminosity, and data rates of video signal presentation device or devices are continually improving. However, a mono head-mounted display screen having 3840×2160 pixel resolution, at 60 frames per second frame rate, and a data input handling rate of 150 megabits per second constant, or omnidirectional 3840×2160 (3840×1080 each eye, arranged top to bottom of the screen) at 60 frames per second frame rate and a data input handling rate of 150 megabits per second constant are well in excess of the minimum requirement. The types of images required to be made available to provide the source human visual presentation information disclosed herein require much less capability than indicated. The field of view of the human wearing the headset incorporating the video signal presentation device or devices can be assessed individually or assumed to apply to most human users.

A human auditory presentation device comprises a housing per ear arranged to provide one or more predetermined non-verbal auditory signals in one embodiment. At least one human non-verbal auditory presentation device is arranged to receive a predetermined non-verbal auditory signal from a controller. The human auditory presentation device further comprises a transducer to convert electrical energy, representative of a non-verbal auditory signal controlled and provided by the controller, into mechanical energy to vibrate the surrounding air. The transducer is located near the ear of the human, the received vibrated air being a representation of the non-verbal auditory signal. Using two human auditory presentation devices permits both ears of a human to be provided with a non-verbal auditory signal. The human auditory presentation device further comprises a housing having the transducer located internal to the casing, wherein the housing is adapted to direct sound generated by the non-verbal auditory transducer into the ear of a human.

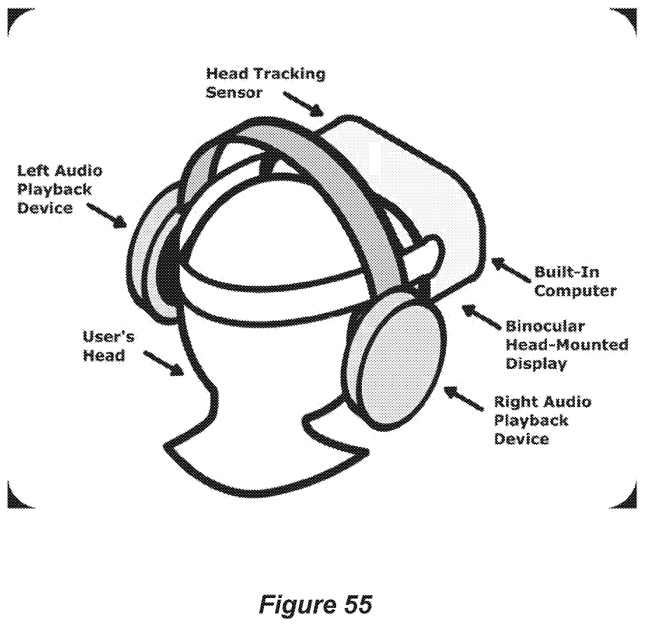

One characteristic but not limited to that is that the visual information presented to the human should be manageable, so visual elements such as menus are not used during the presentation of predetermined visual presentation information, such as, for example, the predetermined alphabet, the predetermined number set to form a human-readable word or number. The Virtual Reality head-worn device of FIG. 55 provides an embodiment of at least one human auditory presentation device having a configuration that exclusively directs non-verbal auditory presentation information in the form of a non-verbal auditory signal to the ear or ears of the human wearing the head-worn device. An alternative is an electrical signal-to-sound transducer located in the arms of a pair of glasses or an in-ear transducer, sometimes called an earbud, of a bone conduction sound conveyance device.

In an aspect of the arrangement, the visual presentation information will be displayed to the user within a human's field of view. When using a Virtual Reality head-worn device, there are eyesight shields and surrounding housing/s to collimate the visual images generated and displayed on the video display screen/s directed towards the eyes of the human wearing the head-worn device. The video display screen is located very close to the eyes of the user. Still, the human field of view can be about 130 degrees for each eye and working together 180 degrees of view wherein the field of view of each eye overlaps by as much as 120 degrees. As long as the visual imaging provided to the user is within their field of view, the user's visual attention will be ensured. Thus, physical barriers and screen areas limit the user's field of view. Areas on the periphery of the field of view may be made available for viewing since there are sensors in the Virtual Reality head-worn devices that allow the user to change the direction of their gaze by, for example, turning their head. The arrangement can be adapted to change the field of view relative to the head movement of the user, and the user's vision, to ensure that the visual information or background being presented on one or more screens of the Virtual Reality device is always within the user's field of view.

It is an aspect of the arrangement that the human-readable source characters or structured sets of characters of one or more alphabets result from source information parsing, meaning that the source sets of characters are partitioned or broken down into individual characters or a number or sets of characters or numbers. A central processor parses visual presentation information stored in the computer server memory and is partitioned to identify words and sentences or subsets of a complete sentence using one or more text spacing elements or punctuation symbols as the delimiter of the word or sentence or a subset of a complete sentence. For example, a space, a comma, semicolon, colon, quotation mark, full-stop, question mark, exclamation mark, and period can all be the end of a word, sentence, or sentence fragment. Using one or more symbols to partition a sentence into smaller fragments would also be possible. A text stream can be further partitioned by identifying individual words or known collections of characters so that in a preferred embodiment, each fragment consisting of one word or symbol can be presented to the human, one at a time, in accordance with the methods described.

For example, information that is in a book that needs to be read and understood by a human, which is typically read by a human at their reading pace, can be presented to that human in a manner in accordance with the methods described and the result may be that the book or relevant parts or passages of the book are read at a much greater rate than that humans reading pace. Still, the retention and perception of the information in the book are greater than if they had read it at the user's most comfortable pace. The same applies to humans in training who need to read information about the safe and responsible approach to the tasks they undertake in their work environment. Likewise, the casual reader of novels can read and appreciate a novel at a much greater reading rate than they usually would while retaining and perceiving details about the novel much better than average.

It is an aspect of the arrangement that the colour of the text is predetermined, as can be a predetermined background colour for the coloured text. For example, the text is white in one embodiment, using a grey background, and the reverse is also possible. Yet further, parsed text can have a predetermined font and colour, wherein those colours comprise four colour palettes that the human user of the arrangement can select from pastel; high chroma for purity, intensity or saturation; primary colours (or secondary or tertiary); and greyscale. By way of example, each fragment consisting of one word or symbol can be presented to the human user in the same colour characteristic, randomly selected or a selected shade from a predetermined colour palette and applied to the collection or each text fragment with a complimentary contrasting background colour. In an embodiment, an arrangement can be set to display text with predetermined colours, improving text reading abilities and retention for human users, particularly those with Dyslexia.

It is an aspect of arranging a predetermined text and background colour. Thus, by way of example, a dark colour, such as black or royal blue, is used as the background to the coloured text to encourage the eye and the mind of the human user to focus on the text only, sometimes referred to as a hyper-focus. In an embodiment, the background colour is predetermined if the human user is a disadvantaged learner (neurodiverse learner). In addition, in an embodiment, the arrangement can provide an option configurable to enable or disable a feature, such as a solid colour laid over the field of view having a predetermined degree of opacity which has advantageous characteristics for neurodiverse human users.

It is an aspect of the arrangement for one character or set of characters representative of a known word to be displayed at a time as text. In essence, the presentation of one word of text at a time. In an embodiment, the human visual presentation device (by way of example, a Liquid-Crystal Display (LCD)) is adapted by size, alignment positioning and distance from the eyes of the human user to present the text as the only visually perceived input within their field of view, being the angular extent of the light received by each eye, unassisted or assisted by the use of a lens or lenses located intermediate the display device and the or each eye or assisted by the use of a light collimator to restrict the view each side of a physically defined area of the display device. The field of view may change with the use of different arrangements, such as when the display is a personal computer (using an application program interface or a web browser), a screen remote from a computing device or serviced by a remote server, or a mobile phone or tablet device. A head-worn apparatus is adapted to collimate the received visual information and direct that visual information to be provided within the field of view of the human. The display device may include a shield to assist the human viewer in confining their field of view to the screen.

The rate of presentation of each word is a characteristic that can be predetermined or changed from a predetermined initial rate according to a predetermined adjusting rate, typically faster the longer the human is using the arrangement or pre-set at a rate more significant than the average reading rate of a particular cohort of humans similar to the human using the arrangement. In an embodiment, it may be that the rate increases at the beginning and reduces near the end of a source of text material (in, say, a page or pages of a document of predetermined training information) to be presented to the human user. The rate can be referred to as words per minute, but that is merely a metric that the human user may best understand. The arrangement measures the rate by characters per set period of the human-readable character or structured set of characters presented or symbols per minute or second. In embodiments, the presentation can present words at a rate that begins at 220 and increases to 1500 words per minute at the beginning of a document and then ramps down the rate, say from 1500 words per minute to 220 words per minute by the end of the document. Of course, the human user can, in an embodiment, select the rate of text display they are most comfortable reading. However, having the rate controlled by the arrangement enables the human user to be challenged regarding their capabilities.

In an aspect of the arrangement, the font size can be an initial predetermined size. Since the size of the visual presentation device is relative to the configuration and location of that device relative to the human user, then the text size is relative to the visual presentation device and its environment. The relative font sizes are in an embodiment, small, medium and large. In an embodiment, the font size can be changed during the presentation of visual information. That variability will enhance the maintenance of the human user's focus on visual information. The human user may be able to select the text size they are most comfortable reading.

In an aspect of the arrangement, the font type can be predetermined since many font types exist. In an embodiment of a font choice option to improve usability for people with Dyslexia, the font OpenDyslexic is available. In an embodiment, the human user may be able to select the font type they are most comfortable reading.

In an aspect of the arrangement, the type of text movement within the field of view of the human user can be predetermined. In an embodiment, the text moves within the field of the human user, and the presentation comprises the human-readable character or structured set of characters appearing to move within the field of view from the human. The movement will be perceived to position the text close to the human perceived position, further from the human within their field of view. The human user may be able to select the rate of movement they are most comfortable reading. It is possible to adjust the text size as well, and human users may be able to select the text they are most comfortable reading.

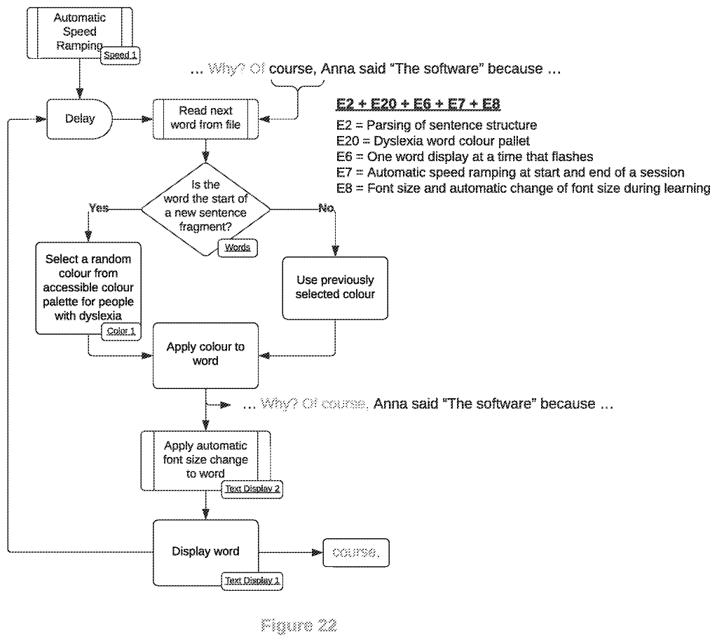

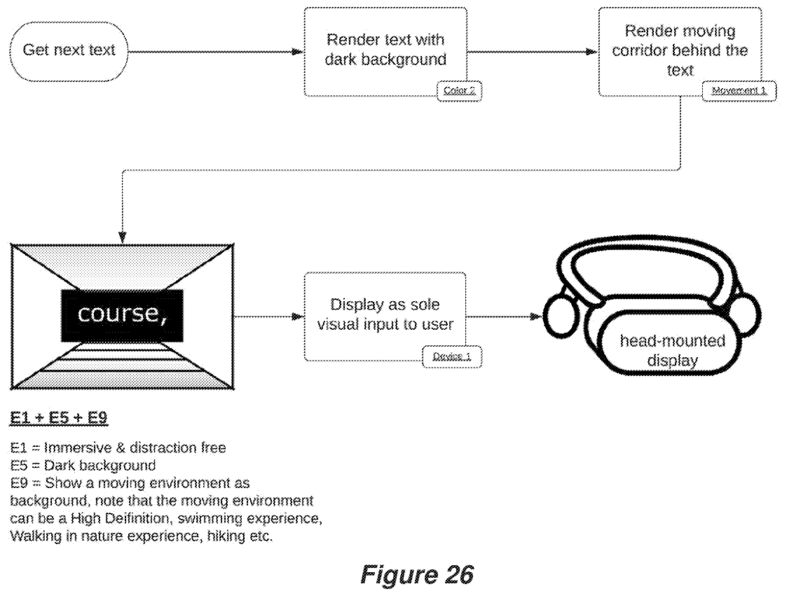

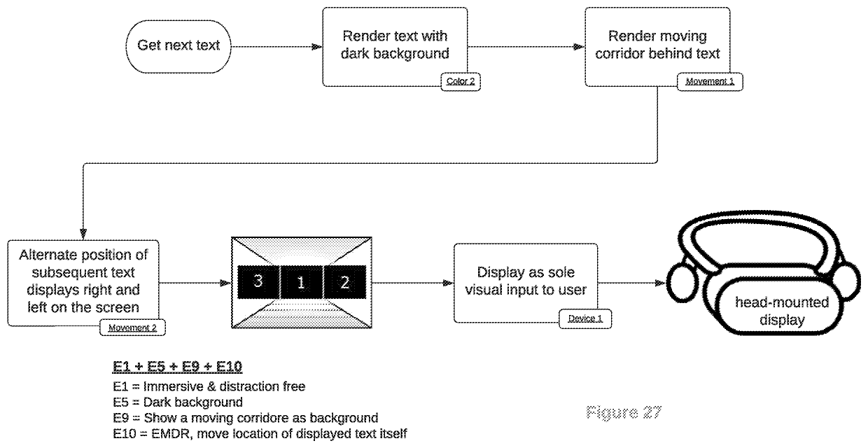

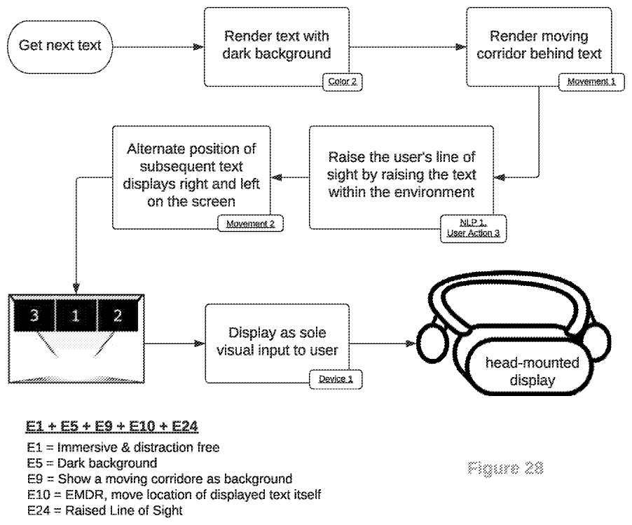

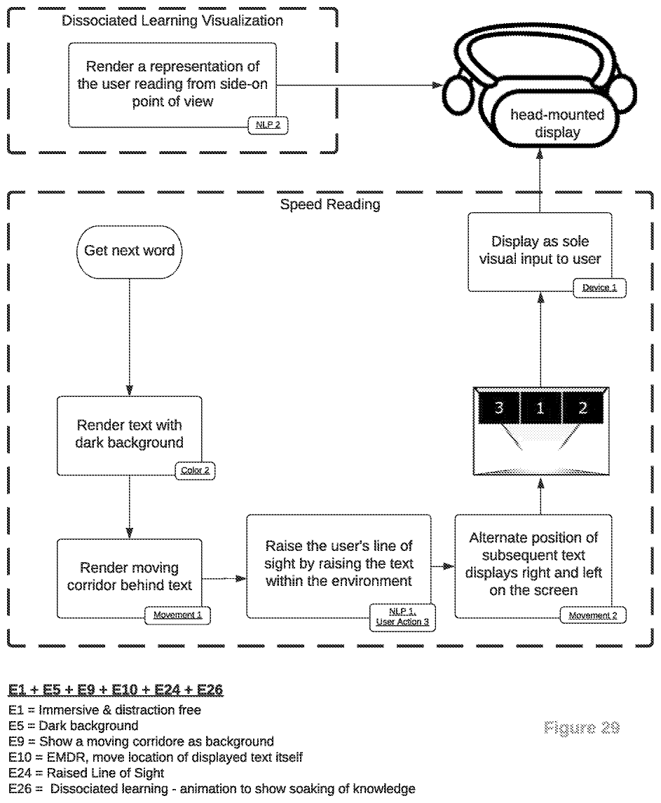

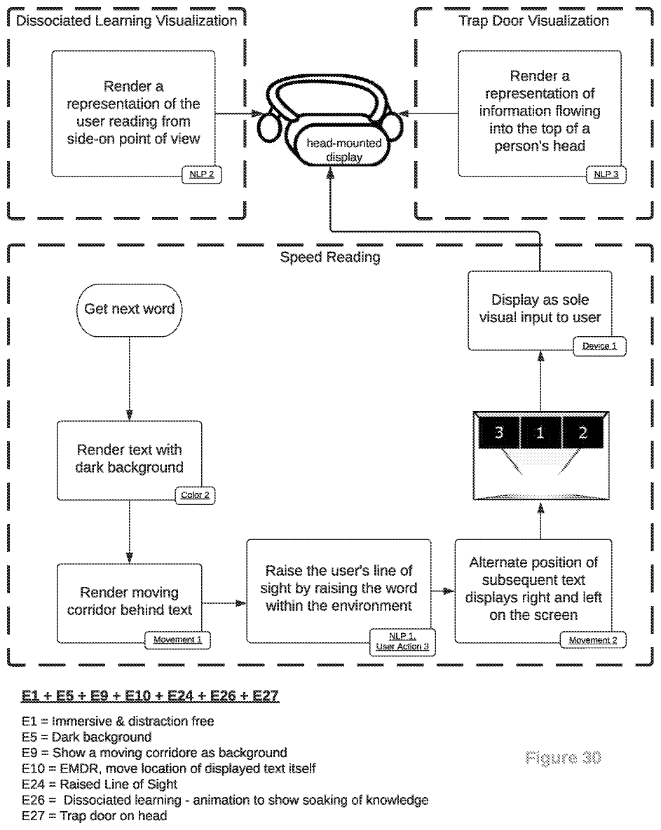

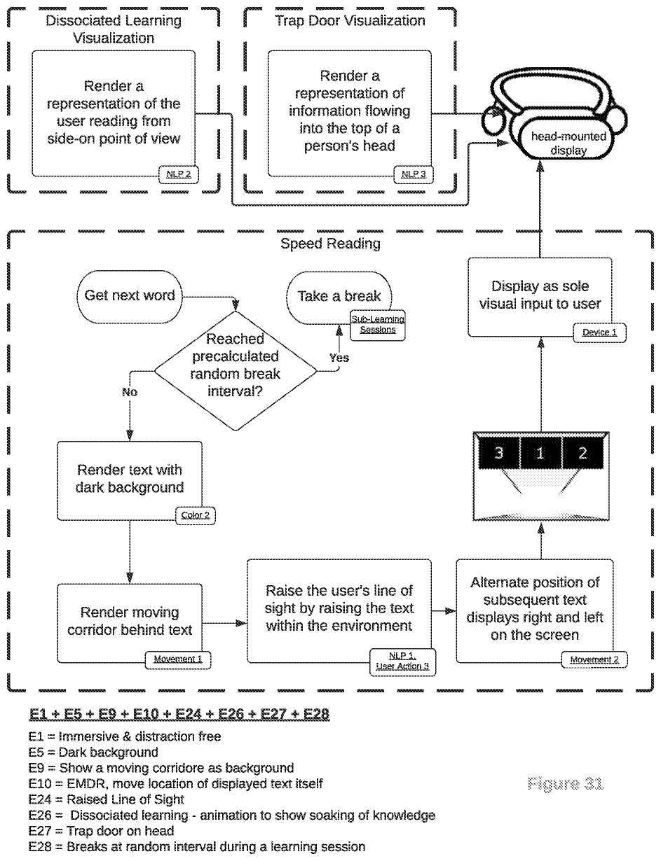

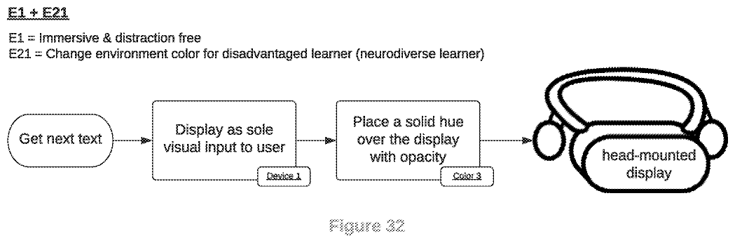

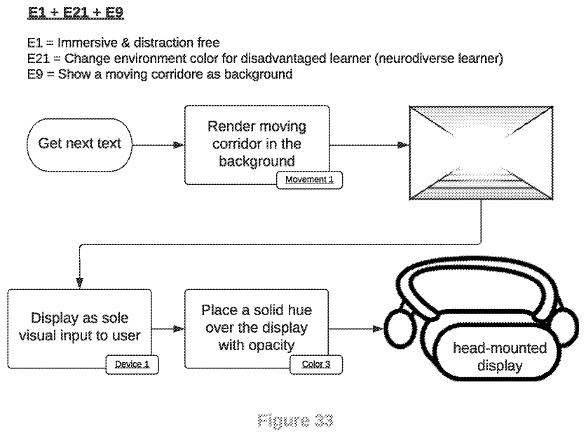

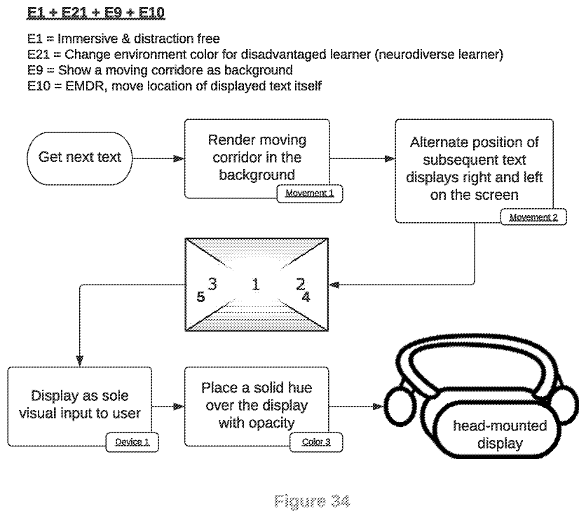

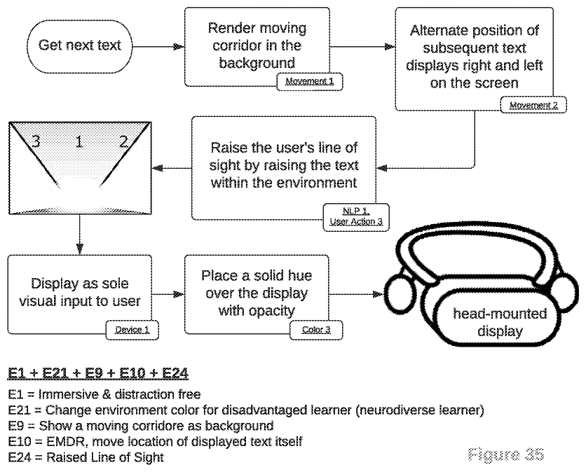

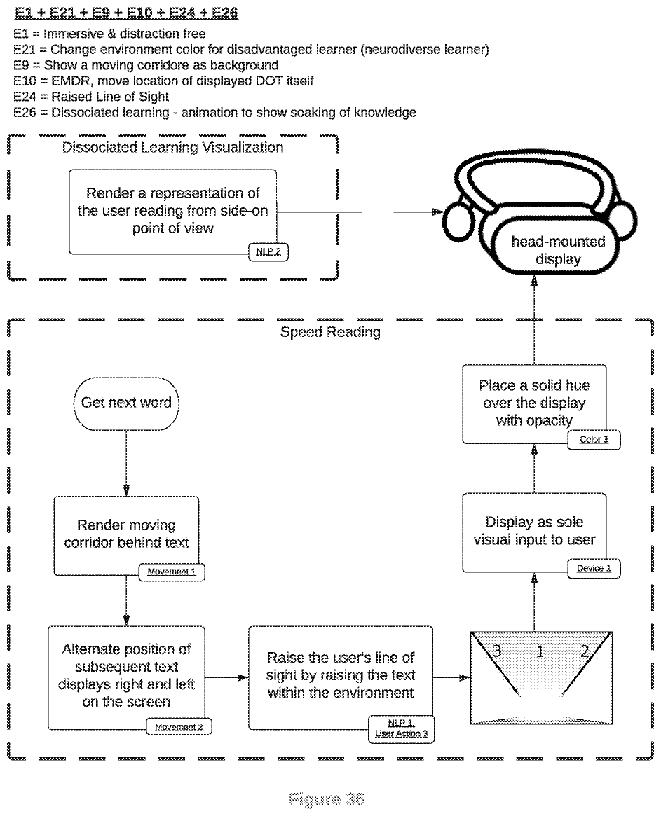

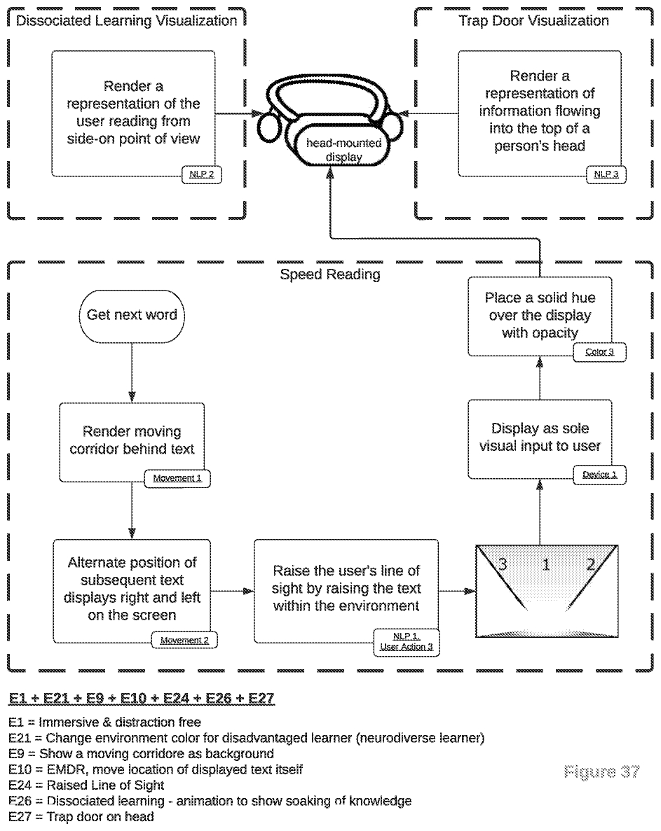

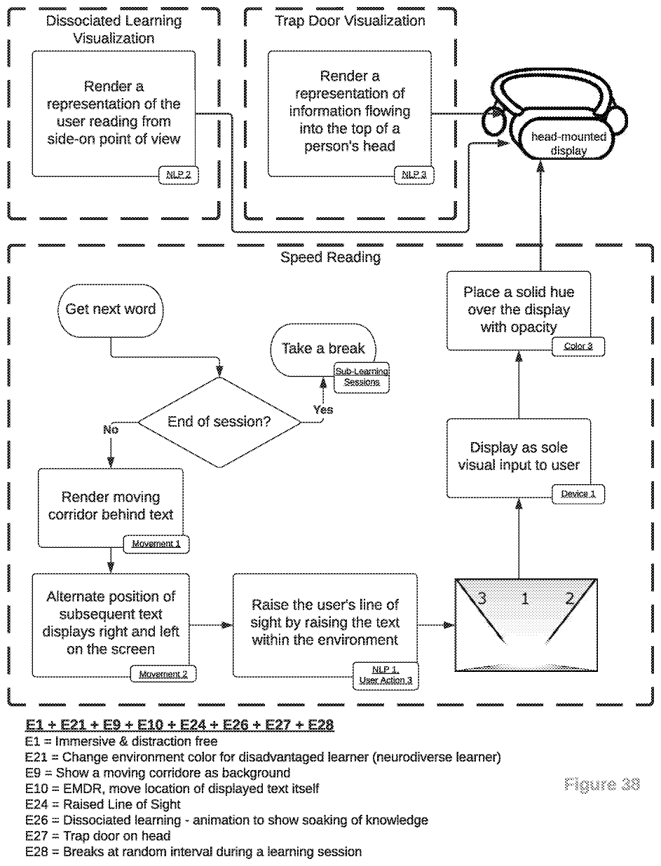

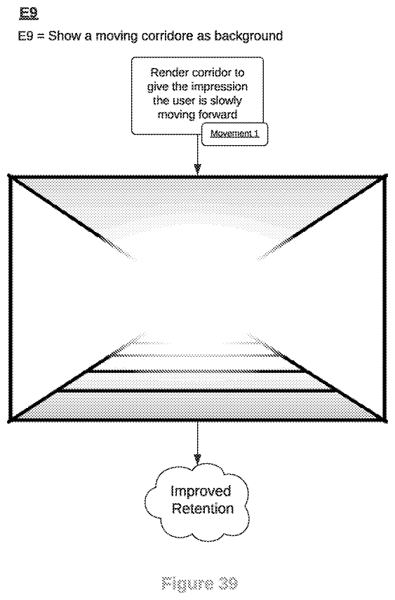

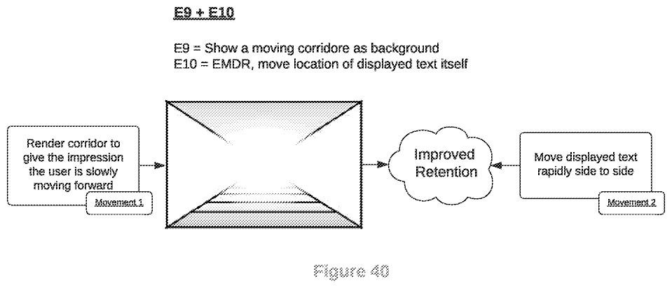

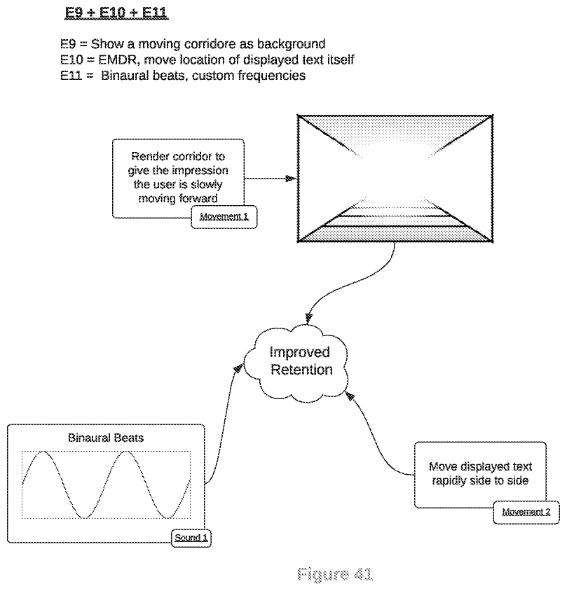

In an aspect of the arrangement, the text's presentation gives the human user the impression that the text is moving forward through a corridor display background (FIGS. 26 to 41 (excluding FIG. 22) provide illustrations of variations of this arrangement) with a more significant movement distance for greater words per minute display rate. The illustration of a corridor to focus the user's attention is but one technique in an array of visual displays, which can include the use of images that mimic a corridor-like environment, such as, for example, a snow skier traversing a long path down a snow-covered hill snaking its way within the snow-covered slopes or between obstacles such as trees. A further visual display could be the line seen by a swimmer doing laps of a never-ending pool lane. Yet again, a never-ending walking trail through a forest or bushland setting. The human user may be able to select the rate of movement they are most comfortable reading. It is possible to adjust the text size as well, and human users may be able to select the text they are most comfortable reading when being presented at a higher rate than they typically read.

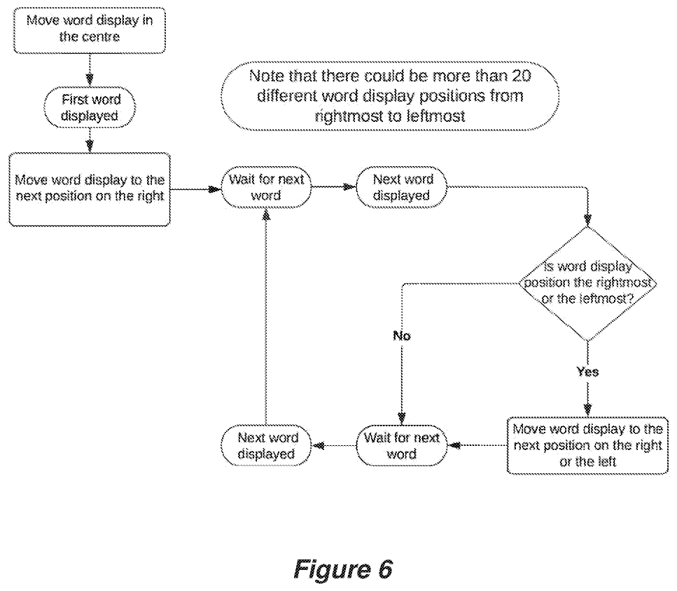

In an aspect of the arrangement, the movement of text within the field of view of the human user can be predetermined. In an embodiment, the text moves within the field of the human user, and the presentation comprises the human-readable character or structured set of characters appearing to move within the field of view from the human perceived position to one side of the human to the other side of the human perceived position within their field of view. FIG. 6 depicts a word positioning process flow chart dealing with word location within a field of view of the user. The embodiment disclosed is one of many text variations presented to the user. One other of the variations is moving the text such that it flows from one side to the other. The movement can be incremental. For example, there are twenty separate increments of movement between one side and the other.

Further, by way of example, the flow can appear to the user to be continuous, which is achieved by small movements which are perceived by a human user as a continuous movement but which are, in fact, multiple small movements at a rate that makes the actual change of position impossible for a human to perceive as anything but continuous. The movement of the text is described as being from one side to the other, but to suit some cultures, the text is moved from the top to the bottom of the field of view. In an embodiment, the presentation of the text provides an impression to the human user that the text is moving location within their field of view from one word to the next. So by way of example, a first word is located at the bottom left-hand side of the field of view, and a second word is located central to the field of view. A third word is located at the top right-hand side of the user's field of view-a more significant rate display of following words for greater words per minute display than they had previously encountered, either before using the arrangement or after some time using the arrangement. Alternatively, the degree and rate of subsequent word presentation may replicate a Rapid Eye Movement (REM) sleep cycle rate. The human user may be able to select the degree and the higher rate of subsequent word presentation they are most comfortable reading in this new reading arrangement and environment.

In an aspect of the arrangement, at least one human auditory presentation device has a configuration that exclusively directs non-verbal auditory presentation information in the form of a non-verbal auditory signal to the ears of one human. For example, using a human auditory presentation device using one or more non-verbal auditory signals, wherein the timing of each human-readable character or structured set of characters is coordinated with the presentation of at least one of a predetermined non-verbal auditory signal. In an embodiment, the coordination is such that the predetermined text and predetermined audio signals are deemed complementary and likely to assist the learning and cognition of the human user. Transitions from one to the other or simultaneous provision of those elements, in their many forms, can be predetermined to suit the user or controlled by the user.

In an aspect, the human auditory presentation device comprises two audio transducers to convert electrical energy into mechanical energy exposed to air. Speakers of suitable size and transducing power capacity are readily used to provide the non-verbal auditory signals used by the apparatus. The speakers may need to be fitted within an enclosure that also covers the ears of the human user. The speakers may be free-standing. As with a Virtual Reality Headset or headphones, the speakers may have wireless communication capability. The predetermined nature of using a non-verbal auditory signal relates to the set-up of the session for use by a human. The type of non-verbal auditory signal selection can depend on the human user's needs during that session. For example, if a human has PTSD, then the frequency of a mono-aural signal will be within a range known to have a desired beneficial psychological effect.

In an embodiment, each predetermined non-verbal auditory signal is a continuous signal of a predetermined frequency or frequency and is to be delivered to the left and right ear as sound waves transduced by the respective speakers. In an embodiment, the sound waves have a predetermined frequency. In embodiments, the frequencies depend on the predetermined words per minute being displayed. In an embodiment, the higher the frequency, the greater the words per minute displayed.

The frequencies include binaural beats, or low-frequency sine waves, intended to induce/replicate deep sleep, REM sleep, relaxation, attentiveness, and resultant assisted cognition.

In an embodiment, the frequencies include 111 hz, the 11th harmonic of the Earth's resonant frequency; the Schaumann Resonance, at 7.83 hz; and predetermined frequencies F1 through F14. Although the LEFT and RIGHT ear are noted below, the reverse case is usable.

$$LEFT EAR F 1 = 111 hz$$

$$RIGHT EAR F 4 = 122 F 5 = 125 F 6 = 128 F 7 = 131 F 8 = 134 F 9 = 137 F 10 = 140 F 11 = 143 F 12 = 146 F 13 = 149 F 14 = 152 F 14$$

In an aspect of the arrangement, a voiceover agent reads the text at the target words per minute. The voiceover agent is, in an embodiment, operable at rates between 100 wpm and 1000 wpm in increments. In an embodiment, the increments are 24 words per minute. In an embodiment, the voice is configurable by the human user to choose the language, gender, or accent of the voiceover.

In an aspect of the arrangement, it is possible to add background mood sounds. In an embodiment, sounds sourced from or representative of sounds from water flowing, waterfall, rainforest, and other sources can be concurrent to any of the one or more described embodiments. The human user may, in an embodiment, indicate a preference for providing a potentially relaxing audio environment. In an embodiment, there may be predetermined audio signals that replicate or are replays of recordings of locations, such as a rainforest, a waterfall, a stream, waves on a beach, or underwater scuba diving. This can be provided in an embodiment before and after the reading arrangement.

In an aspect, the arrangement includes adaption to encourage a human user to use the arrangement with their eyes maximally dilated (eyes wide open state). The user is prompted to open their eyes as wide as possible. This is intended to replicate the state of fear and prompt the fight or flight response to increasing attentiveness.

In an aspect of the arrangement, there is an option for the human user to highlight sections of the text that they wish to mark for future reference. In an embodiment, the arrangement can retain highlighted sections for future reference and possibly re-use by the arrangement. Implementing such a feature may involve the operation of a separate trigger (operable by the human user-touch, voice, physical switch, etc.) which is ON during the display of the text of interest and OFF when the human user is not interested.

In an aspect of the arrangement, there is a display mode referred to as a raised line of sight. The arrangement can provide a configurable option to enable or disable this feature in an embodiment. If enabled, the text/information is always displayed at an angle between 10 and 45 degrees from the human user's horizontal eyesight. The arrangement prompts the human user to raise their head to read the visual information clearly in an immersive environment. Implementing such a feature may involve operating a VR device with head-tracking capability.

An aspect of the arrangement is a learning calendar, including a predetermined schedule to revise previous texts or notes to assist the human user in retaining more of their prior learnings. In addition, the spacing of instruction using the arrangement can be scheduled, and compliance with taking the coursework being instructed can be tracked.

In an aspect of the arrangement, a visualisation tool engenders a dissociated learning state in the human user. In an embodiment of the teachings of such an approach-animation to show a person (not unlike the human user) soaking up knowledge from the course is depicted in the field of view. For example, an animation is available to illustrate a person reading from a side-on view. This image is intended to prompt the user to imagine the user is reading from an out-of-body view, which helps ground the user and focus on the text. In a similar embodiment, using an image or short video animation of a trap door on or in an animated character's head illustrates the top of the head opening up like a trapdoor and information flowing into the head in text form. In an embodiment, a trap door visualisation is provided as an animation showing the top of a person's head opening up like a trapdoor and a visualisation of information flowing into the opening into the head. This imagery is intended to provide the user with the sensation of being in a learning frame of mind, as this animated imagery will enhance the user's focus. An animation is described, but there could be real-time or real-time video enhanced with visual special effects applied post-production of the real-time video.

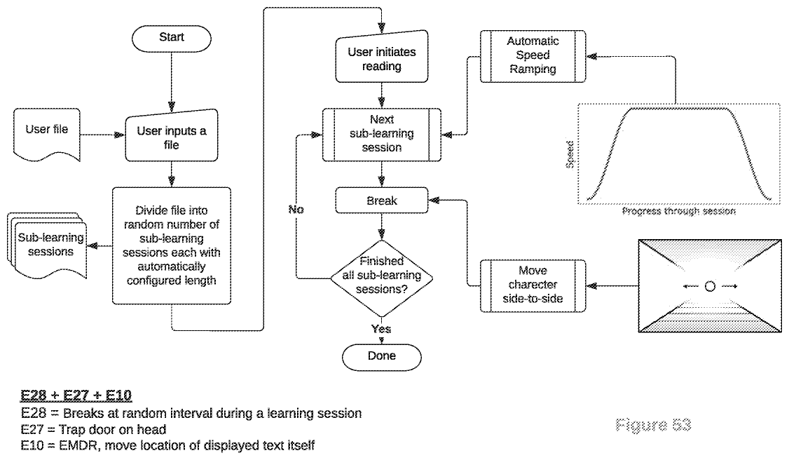

In an aspect of the arrangement, a learning session is segmented into a random number of sub-learning sessions facilitated by creating a break between sub-sessions. In an embodiment, each sub-learning session may start and end with automatic words per minute speed ramping refer to FIG. 53. A predefined configuration determines the duration of each sub-learning session. By way of example only, for each 7 to 10 minutes sub-session of learning, a 30-second to 60-second period of break can be provided to the user. During the break in an embodiment, the visual signal is a black screen, and there may be silence, or there is at least one human auditory presentation device having a configuration that exclusively directs predetermined non-verbal auditory presentation information in the form of a non-verbal auditory signal to the ears of the at least one human. In an embodiment, the audio signal is either monaural or binaural, or a combination of two tones or monaural tones are provided to the ears of the human.

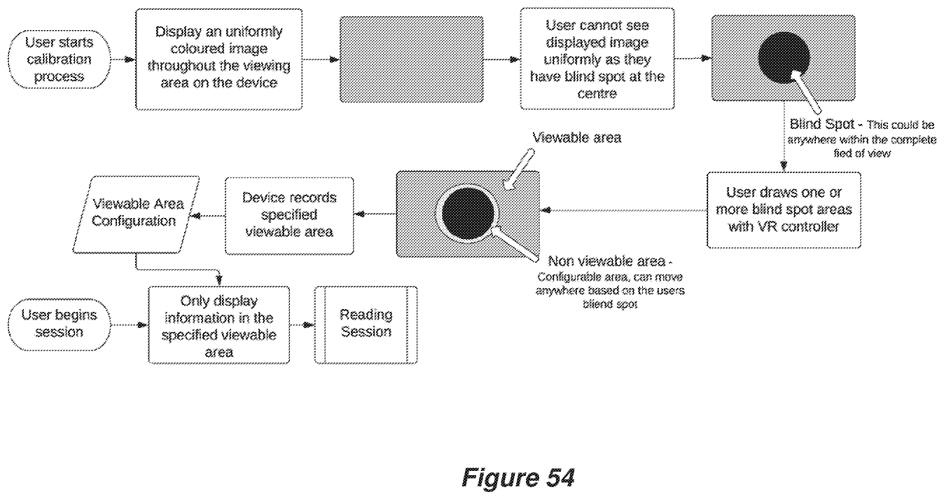

FIG. 54 depicts a calibration process for a head-worn Virtual Reality device and a set-up process depicting the adjustments that may be required if a user has a visual blind spot. The illustration is merely an example, and the blind spot will be of different sizes and possibly shapes depending on the user. The location of the blind spot will also be different for different users, and the forms of compensation for the blind spot can include movement of the test and other imagery into the field of view that remains for a user.

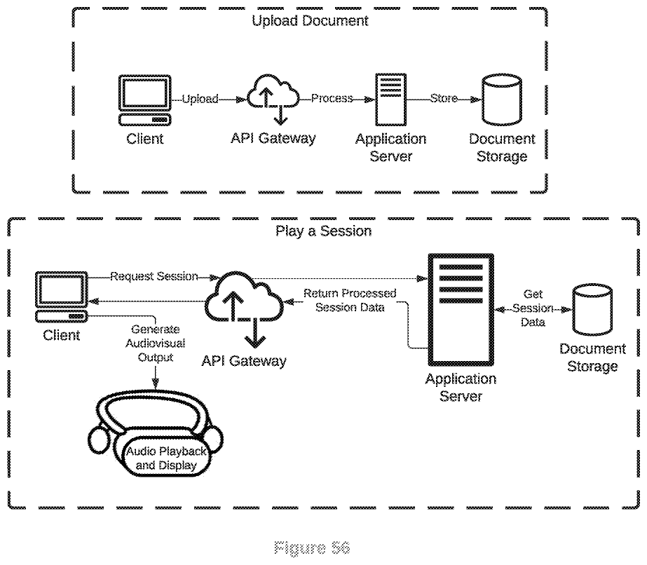

A human sensory information presentation arrangement for presenting sensory information to at least one human is disclosed herein. It comprises a memory controller and a central processing unit programmed to make sensory presentation information available. FIG. 56 depicts one of many possible embodiments. FIG. 56 depicts the Client Server cloud architecture. One function to be performed is the collection and storage of documents which will form the basis of the visual presentation of information to at least one human, as illustrated in the upload of a documented process depicted therein. In an embodiment of a human sensory information presentation arrangement as depicted in FIG. 56, the Client Server cloud architecture provides access to one or more stored documents by an application server which can be located anywhere, as can the document storage. In an embodiment, the client device controlled by the user issues a request to the application server, which is authorised according to Software as a Service agreement between the user (or a nominated third party) to provide a learning session to the client device. A client device can be a mobile phone, a tablet, a personal computer, a computer server and many alternative devices. Such devices have a screen and audio output capability. In the embodiment disclosed, the learning session is delivered to a Virtual Reality headset (FIG. 55) worn by the user/s and sensors embedded therein provide sensor signals to the user client, which may be running a local session of the learning session or provided to the application server which adjusts the delivery of the learning session in accord with the various arrangements disclosed in general terms within this document, such as, raising the user's line of sight. In an embodiment, the application server comprises a controller with a memory and a central processing unit programmed to make sensory presentation information available to at least one human visual presentation device in this embodiment via the client device. In an embodiment, a Virtual Reality head-worn device has a configuration that exclusively directs the visual presentation information to one human user. However, multiple Virtual Reality head-worn devices can be worn by multiple users simultaneously providing the same session.

In an aspect of the arrangement, the visual and aural presentation of information is adapted to support users with impaired vision. In an embodiment, those users with the vision impairment disease “macular degeneration” will be assisted by the Virtual Reality head-worn device's capability to have a set-up calibration process depicted in FIG. 54. Using the set-up process, the user can provide a tangible indication of where in that user's field of vision there is any reduction in the region of that user's perception. The arrangement can then ensure that the presentation avoids using that area or those areas and thus maximises the useable area of the field of vision of that user.

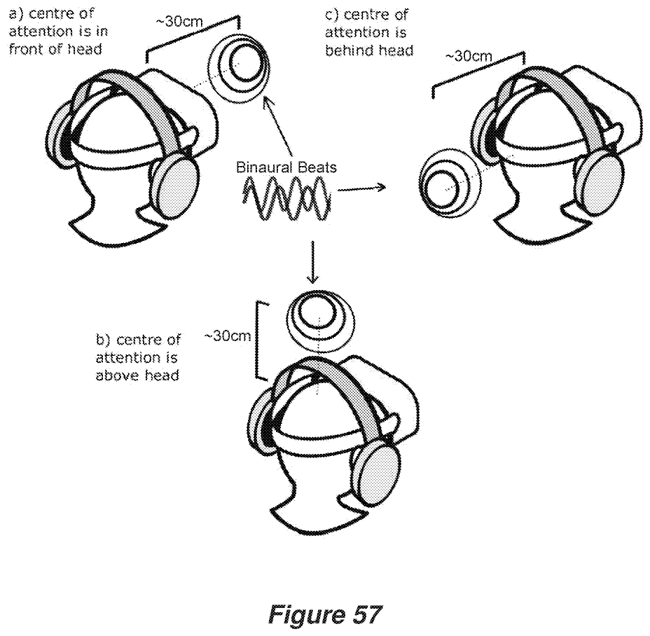

FIG. 57 depicts the use of sound processing techniques to make the sound seem to have a source remote from the user, as illustrated in front, above and behind, at any one time. However, the perceived sound source can be made to be at any location relative to the user, and the illustration of a 30 cm distance is merely an illustration. The technique for generating the required audible signals to product the desired outcome is known to those of skill in that art. This embodiment provides a technique for focusing the user's mind while providing the sound and imagery described in this document in various forms.

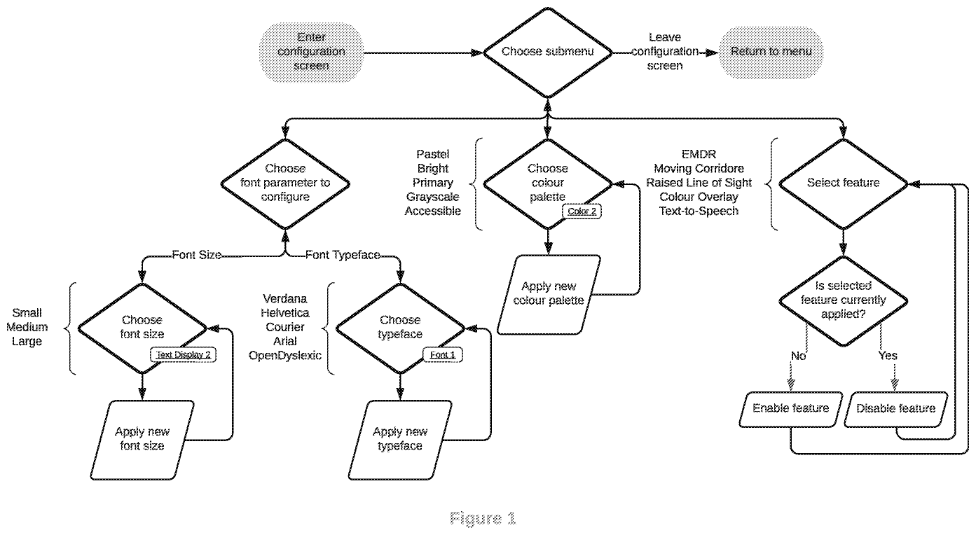

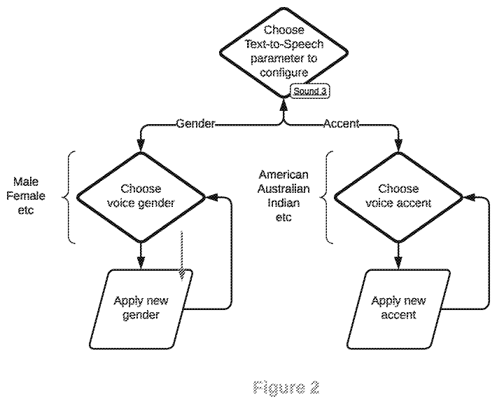

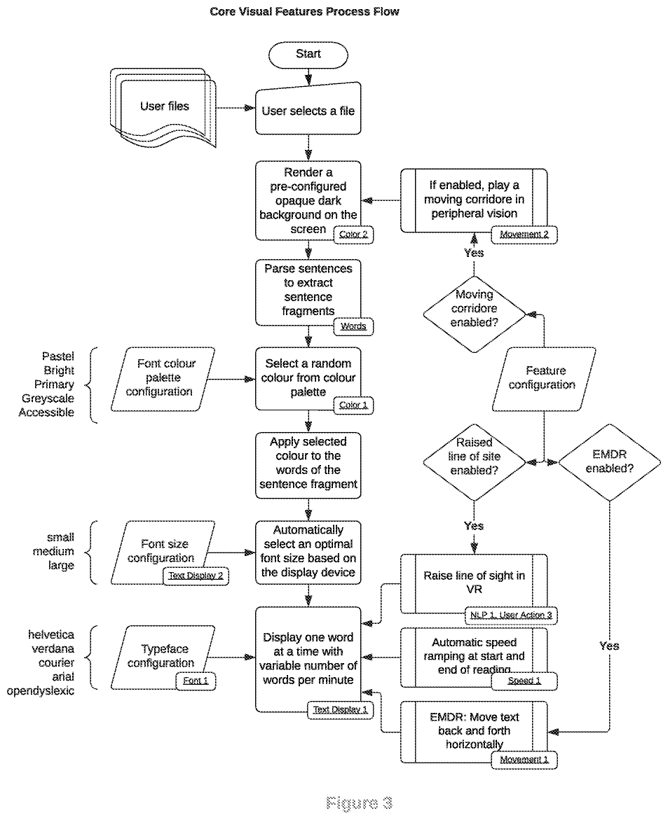

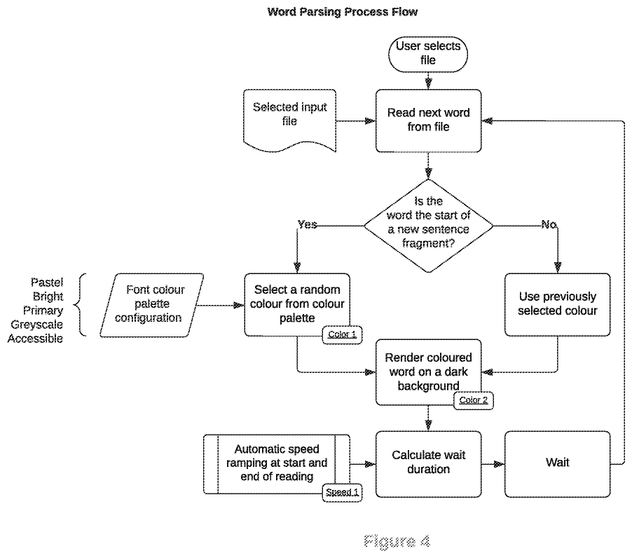

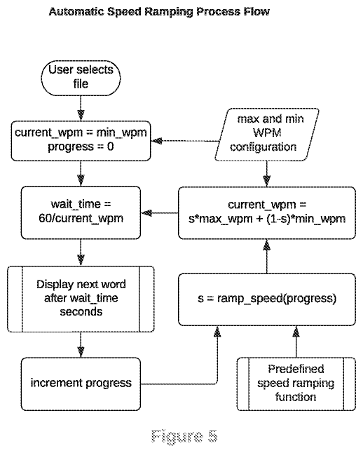

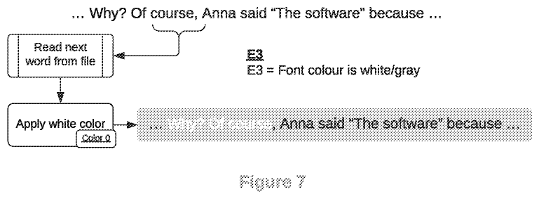

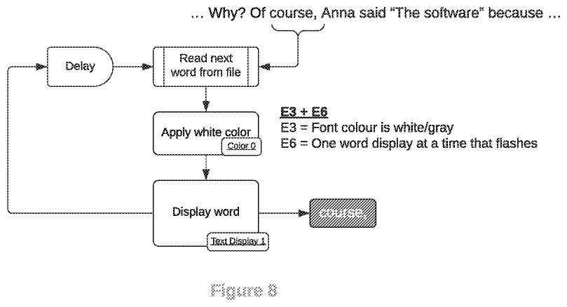

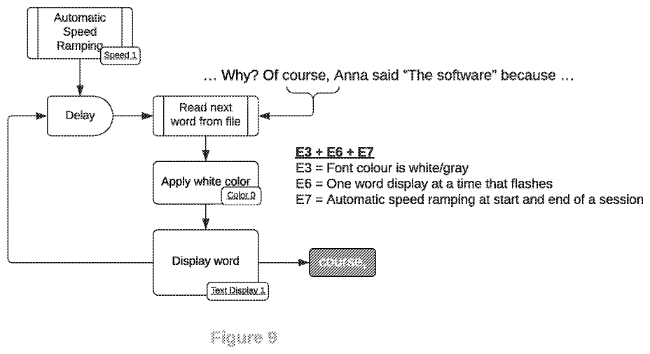

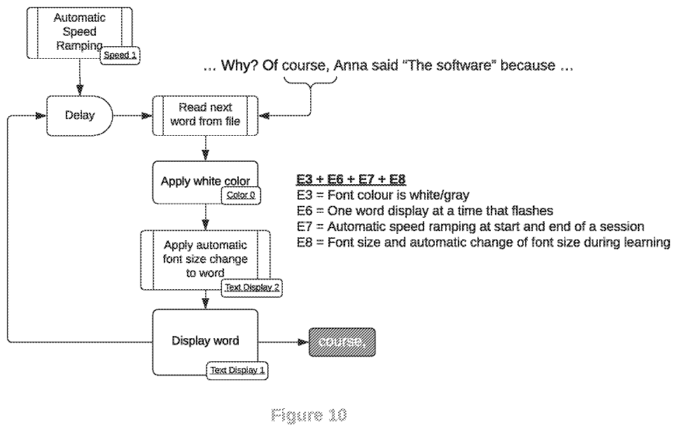

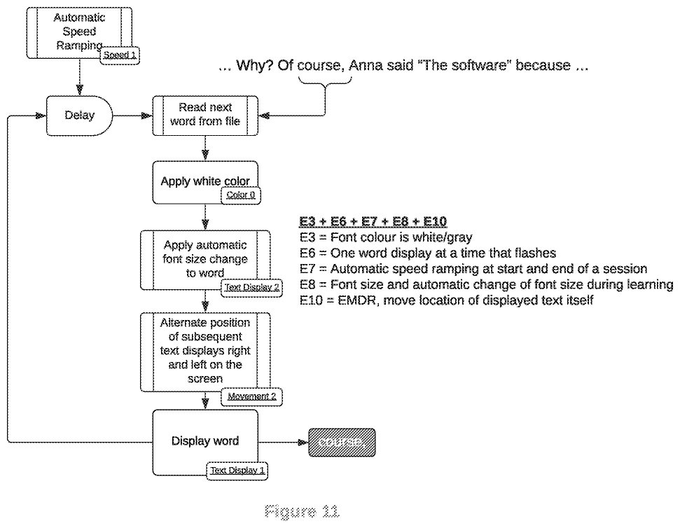

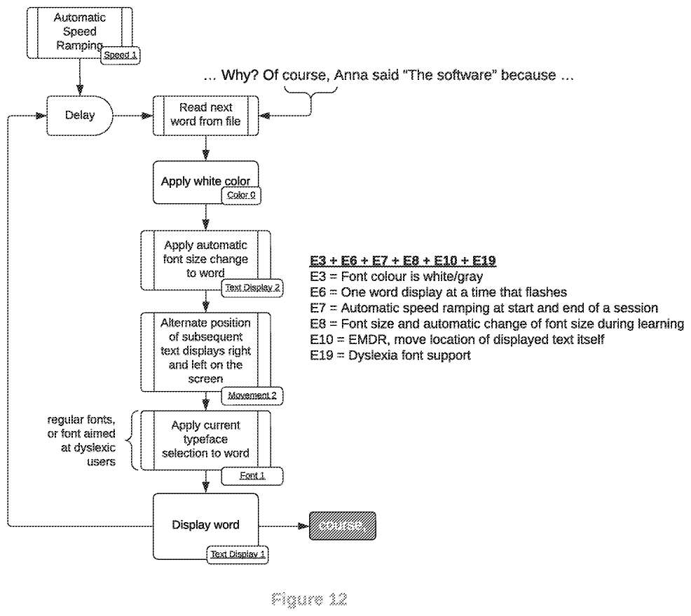

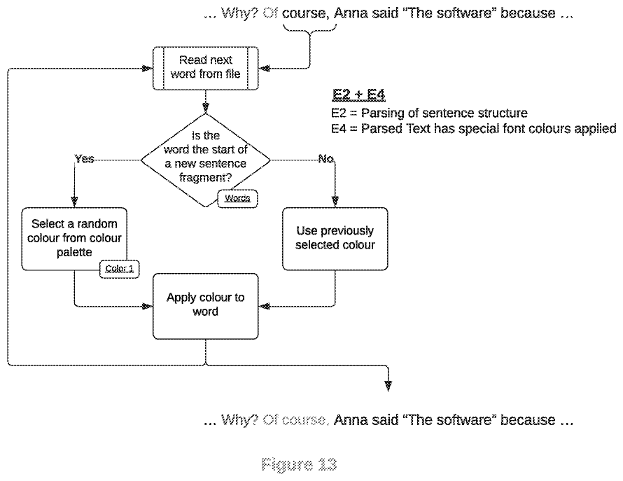

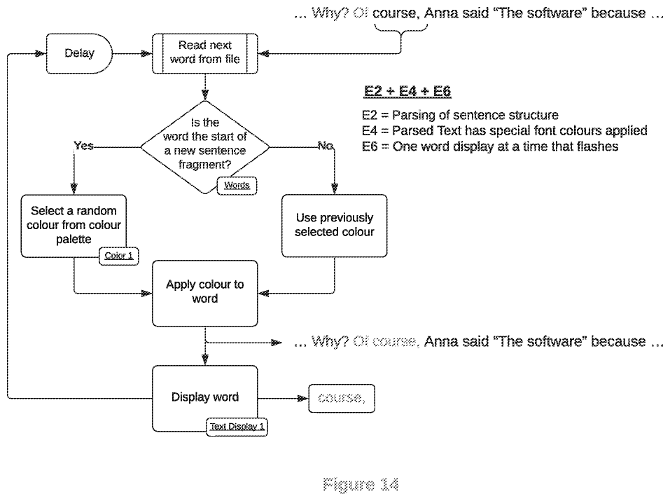

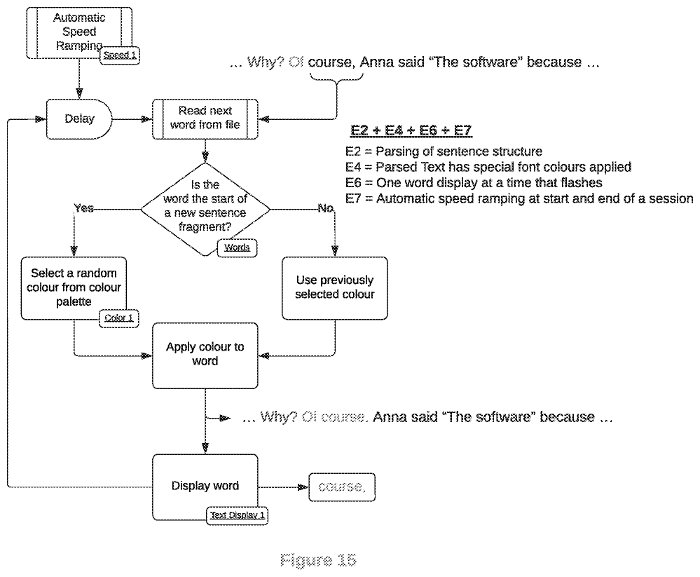

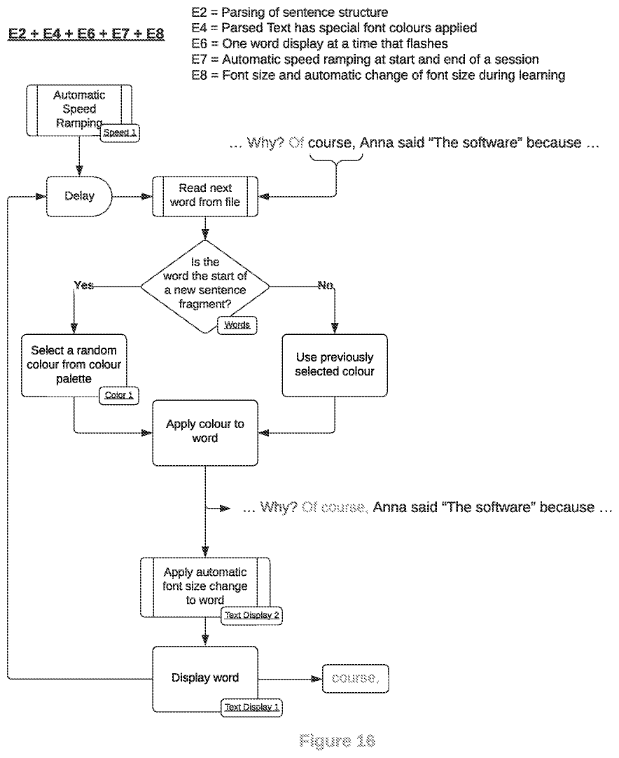

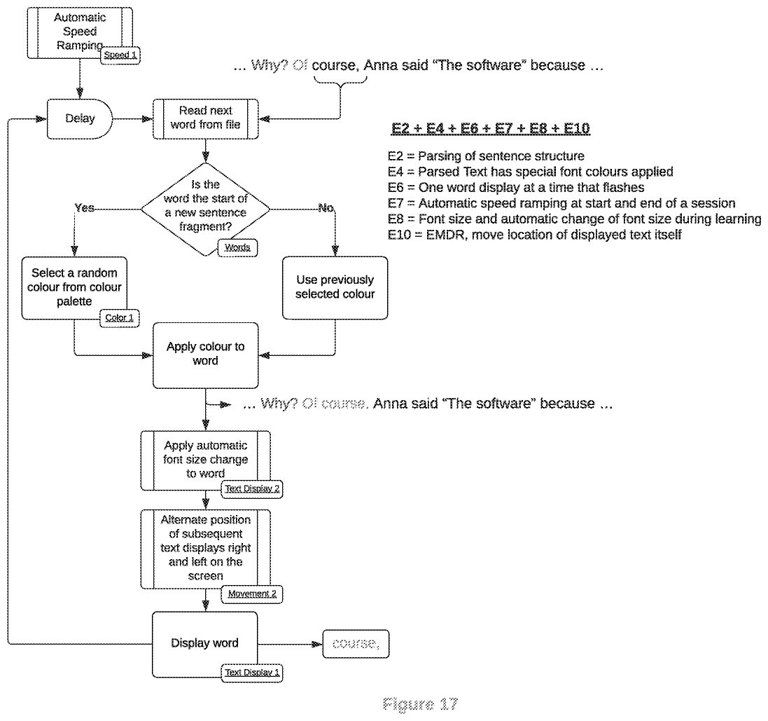

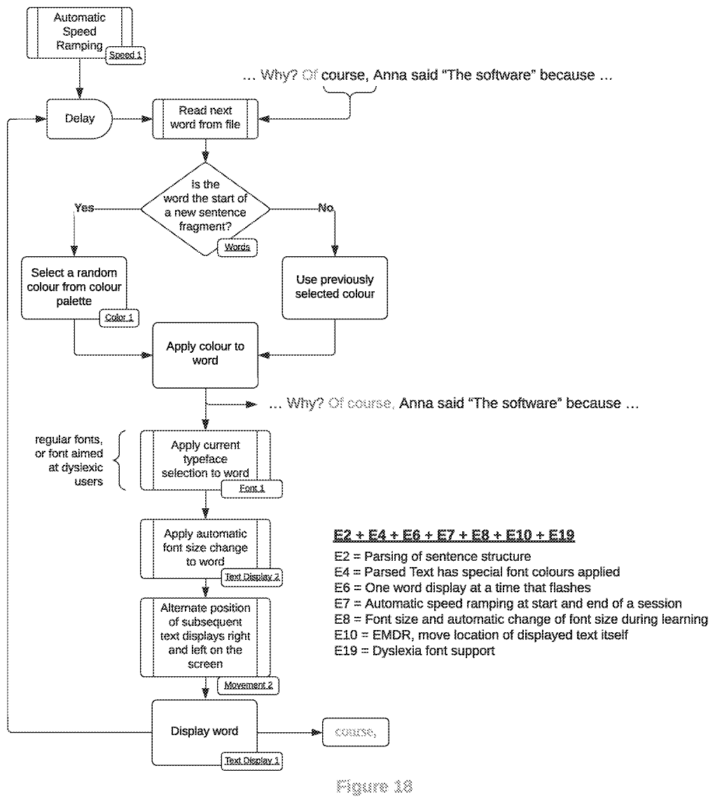

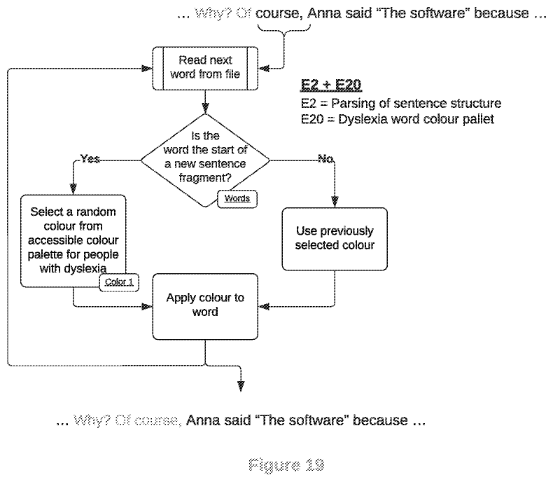

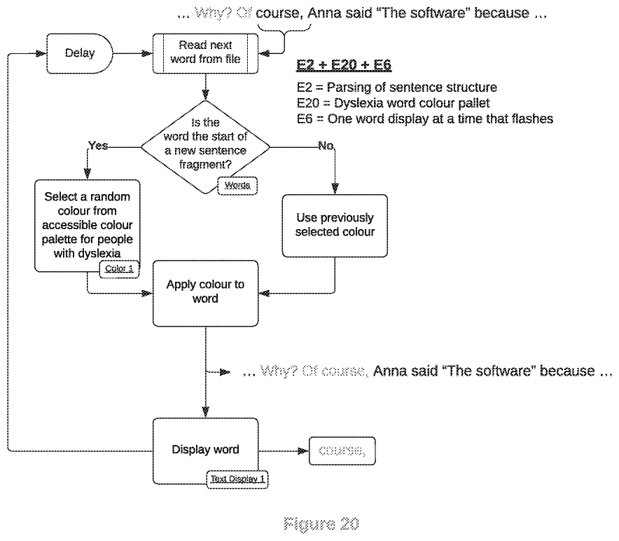

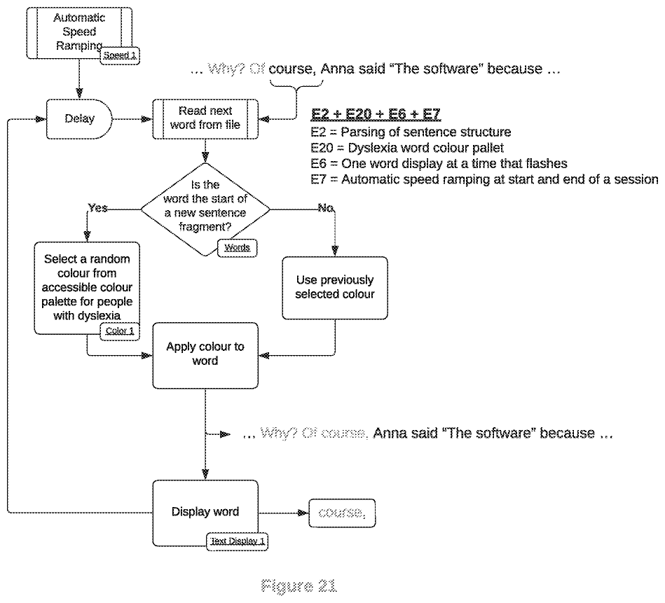

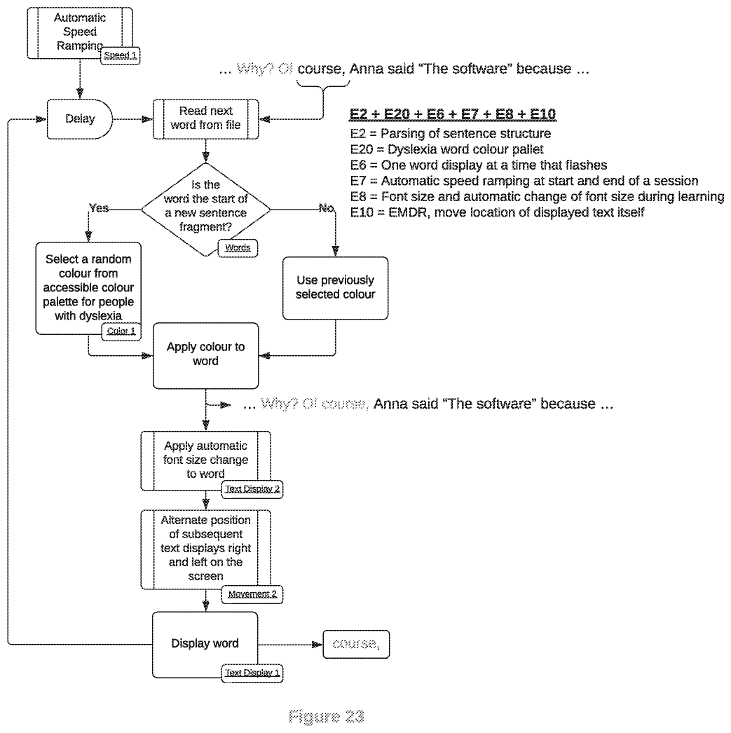

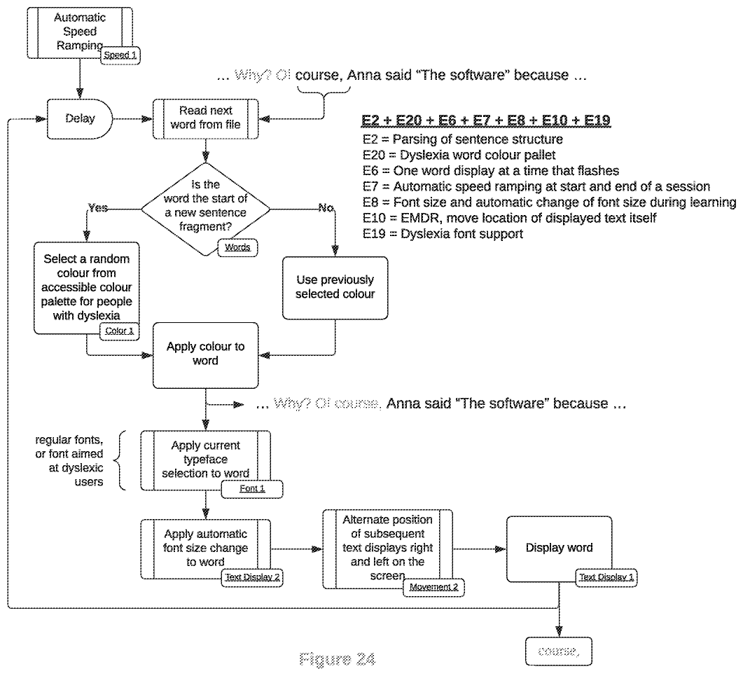

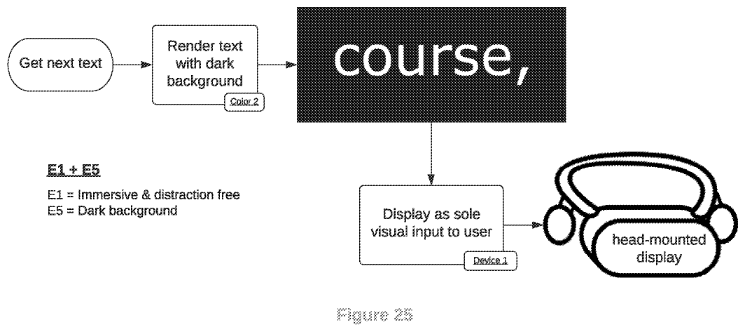

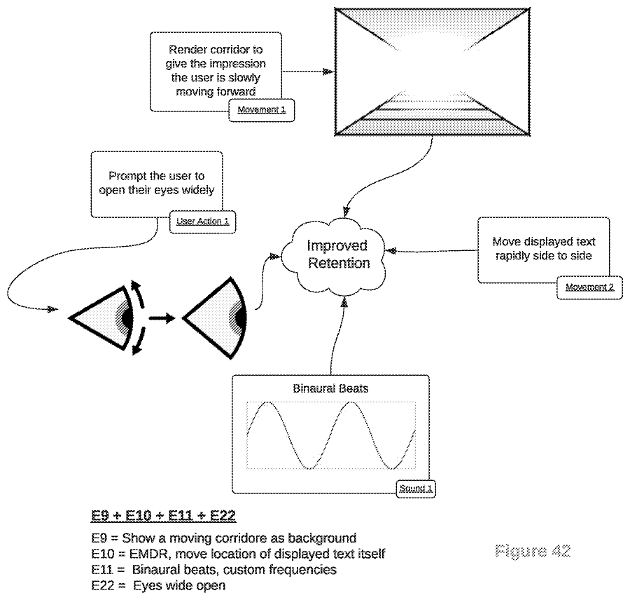

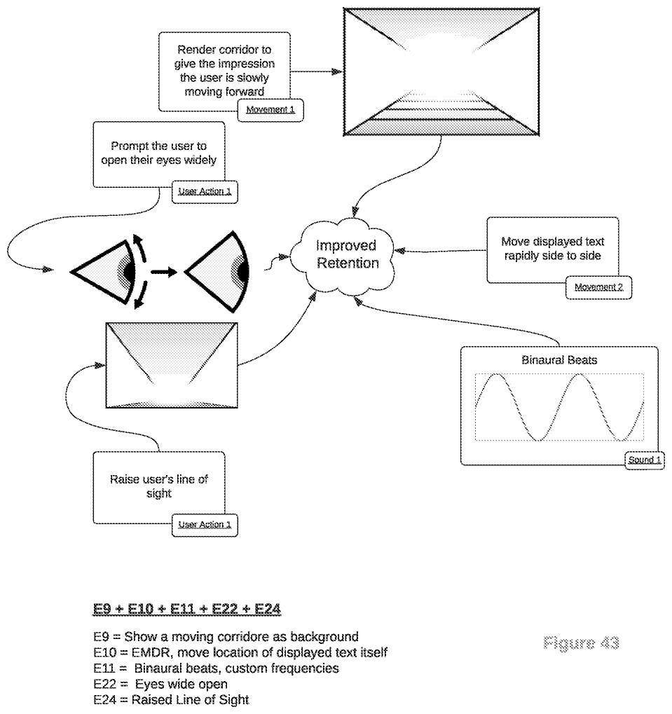

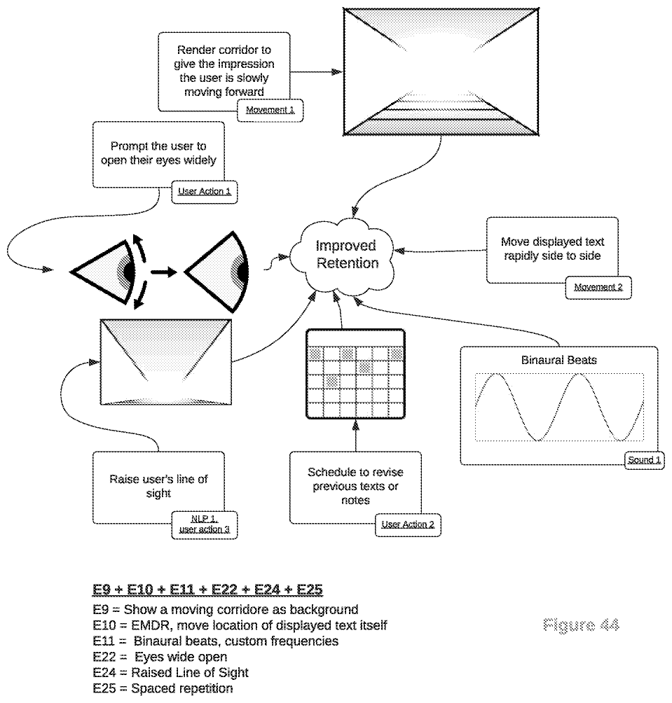

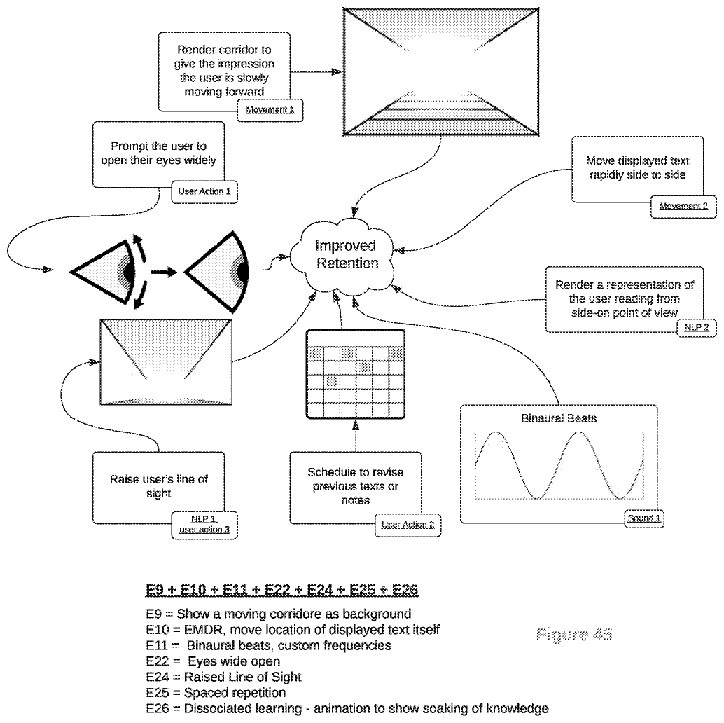

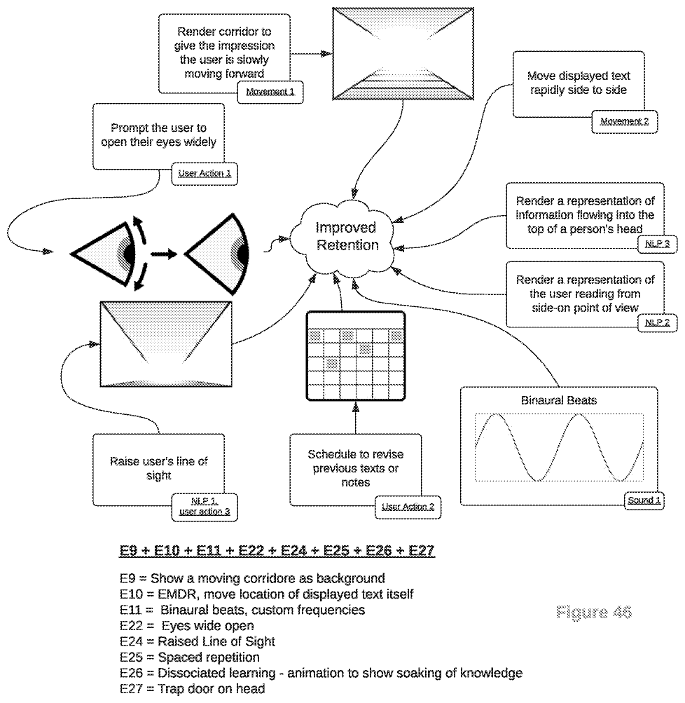

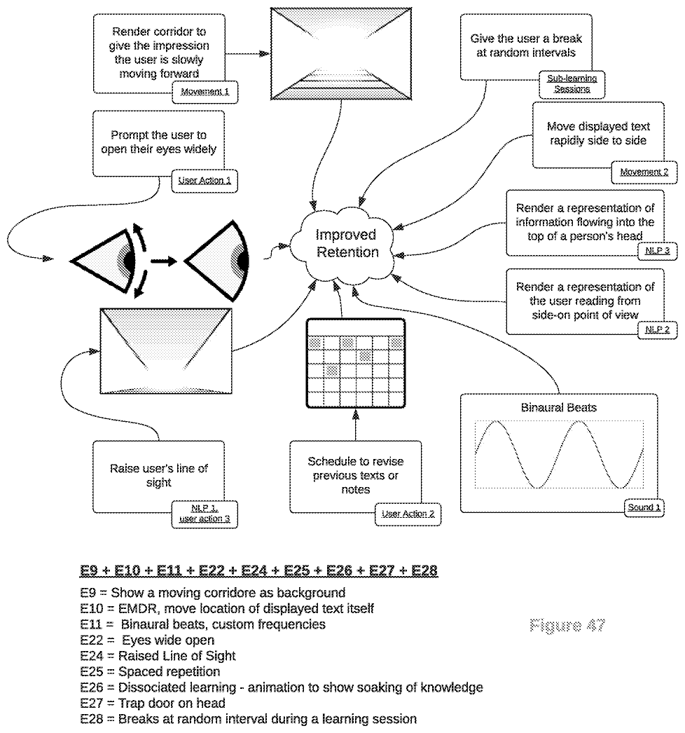

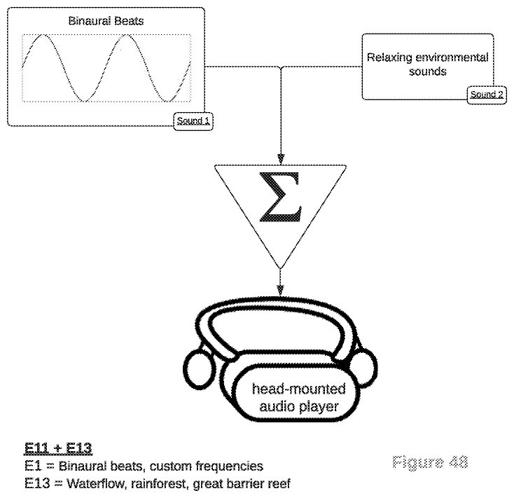

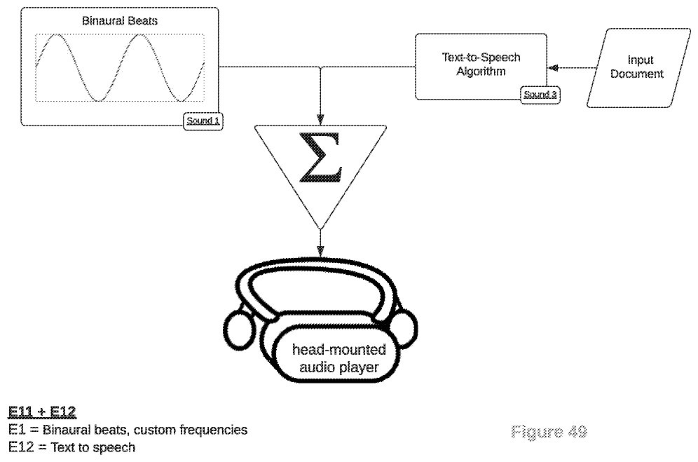

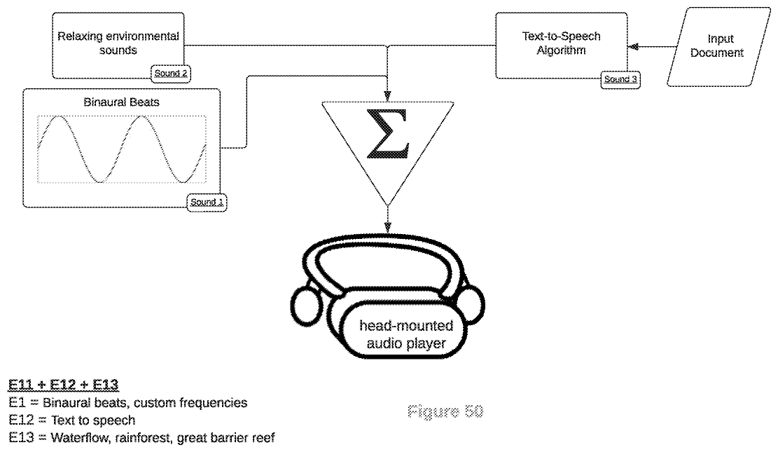

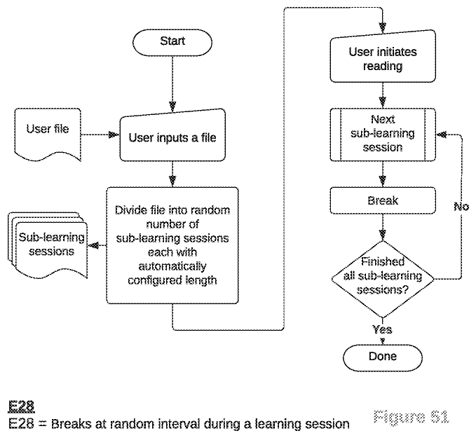

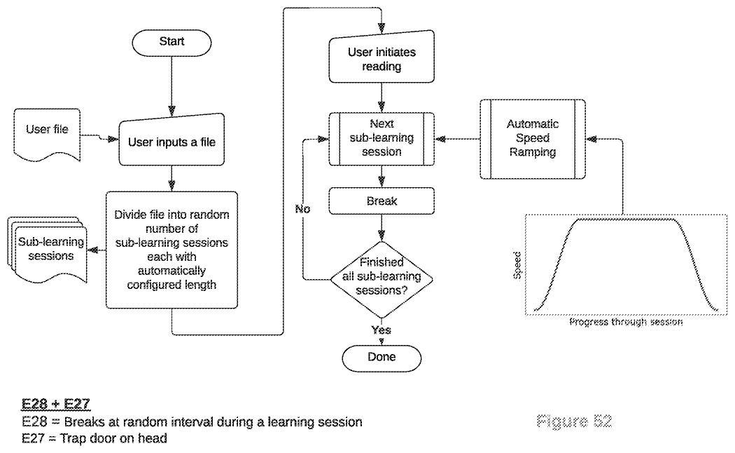

Each of FIGS. 1 to 57 includes details of various embodiments. However, the embodiments displayed are not the only embodiments of the various elements disclosed in those figures. There are many combinations of each of the features disclosed, which are not displayed, but teaching the various combinations provides the basis for using different combinations. Furthermore, different combinations may be beneficial to one or more users. As the development process is implemented and used by various users with different mental acuity and cogitative capabilities evolves, certain combinations will be more effective than others. Some users are also expected to benefit from using the apparatus and processes as disclosed. Thus, those users with improving abilities may require different learning stimuli and information to further their progress involving other combinations of the various elements disclosed. Yet further, there will be applications of the techniques disclosed and taught herein in areas yet to be contemplated.

1. A human sensory information presentation arrangement for presenting sensory information to a human, the arrangement comprising:
a controller comprising a memory and a central processing unit programmed to make available human visual and auditory presentation information, including at least visual information and non-verbal auditory signals;
at least one human visual presentation device for receiving visual information from the controller, the visual presentation device having a configuration that exclusively displays the received visual presentation information to the one human within a predetermined field of view, wherein the visual information comprises at least one or more human-readable characters or structured set of characters from a predetermined alphabet and predetermined number set to form a human-readable word or a number, presented one character or one structured set of characters at a time; and
at least one human auditory presentation device for receiving a non-verbal auditory signal from the controller, the human auditory presentation device having a configuration that exclusively directs the non-verbal auditory signal to the one human, the non-verbal auditory signal comprising one or more non-verbal auditory signals,
wherein the controller presents each human-readable character or structured set of characters while simultaneously presenting at least one of a predetermined non-verbal auditory signal.

2. The human sensory information presentation arrangement of claim 1 wherein the human-readable characters or structured set of characters are presented by the visual presentation device to contrast with a predetermined background colour also presented by the visual presentation device.

3. The human sensory information presentation arrangement of claim 1 wherein the visual presentation of a human-readable character or structured set of characters includes one or more of the group: a predetermined colour for one or more of the human-readable characters or the structured set of characters; a grouping of different coloured human-readable characters or structured set of characters; the human-readable character or structured set of characters that are adapted to appear to move in front of the human viewing the presentation.

4. The human sensory information presentation arrangement of claim 1 wherein a predetermined colour is used for a predetermined word and to contrast with a predetermined different colour to that of the predetermined word.

5. The human sensory information presentation arrangement of claim 1 wherein predetermined colours are used when a series of presented words form a human-readable phrase or a human-readable sentence.

6. The human sensory information presentation arrangement of claim 1 wherein the visual presentation comprises the human-readable character or structured set of characters that appear to move close to the human and then further from the human within their field of view, wherein the movement coincides with the appearance of each successive human-readable character or structured set of characters or during the appearance of a successive human-readable character or structured set of characters.

7. The human sensory information presentation arrangement of claim 1 wherein the rate of the successive presentation of a human-readable character or structured set of characters presented to the human is changeable from an initial predetermined rate.

8. The human sensory information presentation arrangement of claim 1 wherein the font size of the successive presentation of a human-readable character or structured set of characters presented to the human is changeable from an initial predetermined font size.

9. The human sensory information presentation arrangement of claim 1 wherein the successive presentation of a human-readable character or structured set of characters presented to the human with an image of a trap door and the prior human-readable character or structured set of characters presented to the human appears to enter and disappear into the trap door.

10. The human sensory information presentation arrangement of claim 1 wherein the successive presentation of a human-readable character or structured set of characters presented to the human with an image of a moving corridor and the prior human-readable character or structured set of characters presented to the human appears to enter and disappear into the corridor.

11. The human sensory information presentation arrangement of claim 1 wherein the successive presentation of a human-readable character or structured set of characters presented to the human with repetition of one or more of the human-readable character or structured set of characters.

12. The human sensory information presentation arrangement of claim 1 wherein each predetermined non-verbal auditory signal is continuous during the presentation of a human-readable character or structured set of characters and between the successive presentation of a human-readable character or structured set of characters.

13. The human sensory information presentation arrangement of claim 1 wherein one predetermined non-verbal auditory signal is monaural and different to another monaural predetermined non-verbal auditory signal, both provided independently to the human by the human auditory presentation device.

14. The human sensory information presentation arrangement of claim 1 wherein the predetermined non-verbal auditory signal is binaural and provided to the respective ears of the human.

15. The human sensory information presentation arrangement of claim 1 wherein a predetermined non-verbal auditory signal is a combination of two tones, and monaural tones are provided to both the ears of the human.

16. The human sensory information presentation arrangement of claim 1 wherein a predetermined non-verbal auditory signal is isochronic tones.

17. The human sensory information presentation arrangement of claim 16 wherein the isochronic tones have a predetermined pitch and a predetermined interval.

18. The human sensory information presentation arrangement of claim 1 wherein at least one human auditory presentation device for receiving a non-verbal auditory signal from the controller further comprises a transducer to convert electrical energy, representative of a non-verbal auditory signal controlled and provided by the controller, into mechanical energy to vibrate the surrounding air, the transducer being located near an ear of the human, the received vibrated air being a representation of the non-verbal auditory signal.

19. The human sensory information presentation arrangement of claim 17, wherein at least one human auditory presentation device further comprises a housing having the transducer located internal to the casing, wherein the housing is adapted to direct sound generated by the non-verbal auditory transducer into an ear of the human.

20. The human sensory information presentation arrangement of claim 1, wherein the presentation of human-readable characters or structured sets of characters is coordinated with the presentation of a predetermined non-verbal auditory signal by an application programming interface executed by the controller.

21. The human sensory information presentation arrangement of claim 1, wherein the human visual presentation device comprises one video signal presentation screen or two video signal presentation screens that extend at least to the boundary of the field of view of the human.

22. A human sensory information presentation system comprises:
a computer server having a computer server memory and a central processing unit adapted to make available visual and non-verbal auditory presentation information from the computer server memory;
a computer device having a digital signal receiving arrangement, a computer device memory and a central processing unit adapted to store an application programming interface in the computer device memory and execute the application programming interface, which is adapted to receive and process visual and non-verbal auditory presentation information made available by the computer server; and
at least one human visual presentation device for receiving visual presentation information from the computer device, the visual presentation device having a configuration that exclusively displays the received visual presentation information to the one human user within a predetermined field of view, wherein the visual information comprises at least one or more human-readable characters or structured set of characters from a predetermined alphabet and predetermined number set to form a human-readable word or a number, presented one character or one structured set of characters at a time; and
at least one human auditory presentation device for receiving a non-verbal auditory signal from the computer device, the human auditory presentation device having a configuration that exclusively directs the non-verbal auditory signal to the one human user, the non-verbal auditory signal comprising one or more non-verbal auditory signals, wherein the computer device presents each human-readable character or structured set of characters while simultaneously presenting at least one of a predetermined non-verbal auditory signal.

23. The human sensory information presentation system according to claim 22, wherein the visual presentation information is made available to the computer server and stored in the computer server memory.

24. The human sensory information presentation system according to claim 22, wherein the central processor parses visual presentation information stored in the computer server memory being partitioned to identify words and sentences or subsets of a complete sentence using one or more text spacing elements or punctuation symbols as the delimiter of the word or sentence or a subset of a complete sentence.
